(19) United States
(12) Patent Application Publication
(10) Pub. No.: US 2026/0283475 A1
(43) Pub. Date: Sep. 24, 2026

(54) GRIDEYE SENSOR

(71) Applicant: Key Tronic Corporation, Spokane Valley, WA (US)

(72) Inventors: Chad Orebaugh, Liberty Lake, WA (US); Craig Gates, Mead, WA (US)

(21) Appl. No.: 19/084,417

(22) Filed: Mar. 19, 2025

(51) Int. Cl. A61B 5/01; A61B 5/00; G01K 13/20

(52) U.S. Cl. CPC A61B 5/015; A61B 5/6817; A61B 5/742; G01K 13/223; A61B 2562/0271

(57) ABSTRACT

The disclosed advanced ear infection detection device enhances diagnostic accuracy and efficiency, particularly for acute otitis media. A grid array infrared sensor offers both contact and non-contact options while integrating traditional temperature measurements. The device employs algorithmic calibration and data analytics to refine detection accuracy, ensuring precise, real-time assessments. Its wide-surface infrared scanning improves data collection, reducing diagnostic errors and streamlining the process. With rapid, reliable readings in seconds, it minimizes patient discomfort and seamlessly integrates into various healthcare settings, including pediatrics and emergency care. By addressing the limitations of conventional methods, this innovation sets a new standard in medical diagnostics, leveraging technology to improve.

### BACKGROUND

Acute otitis media (AOM), a common ear infection, causes inflammation, pain, fever, and hearing difficulties, particularly in children. Traditional diagnostic methods, such as otoscopes and manual inspections, are often subjective and reliant on practitioner skills, leading to inconsistencies. Additionally, invasive assessments can cause discomfort, highlighting the need for a more precise and patient-friendly solution.

This novel ear infection detection device integrates a grid array infrared sensor and traditional temperature measurement capabilities to eliminate positional inaccuracy. The grid array captures data from a broader area, while traditional temperature sensing ensures precise readings even if the device is not perfectly positioned. Advanced algorithms process sensor data, compensating for variations in positioning and ambient conditions to enhance diagnostic accuracy.

With its non-intrusive design, real-time monitoring, and wireless connectivity, the device seamlessly integrates into modern healthcare systems. Its adaptability to environmental changes sets a new standard in ear infection diagnostics, improving patient care and medical efficiency.

### BRIEF DESCRIPTION OF THE DRAWINGS

The detailed description is set forth below with reference to the accompanying figures. In the figures, the left-most digit(s) of a reference number identifies the figure in which the reference number first appears. The use of the same reference numbers in different figures indicates similar or identical items. The systems depicted in the accompanying figures are not to scale and components within the figures may be depicted not to scale with each other.

FIG. 1 illustrates an external isometric view of the ear infection detection device, showcasing its key components, including the sensor array, housing structure, and user interface elements such as the display screen and control buttons.

FIG. 2 provides a perspective view of the device, emphasizing its external components and their interaction with the ear's tympanic membrane. This figure also illustrates how diagnostic data is displayed through the user interface and transmitted via wireless connectivity solutions.

FIG. 3 depicts an isometric exploded view of the device, specifically highlighting the grid array sensor and the accompanying speculum, which encases the sensor in the final assembled configuration.

FIG. 4 presents a detailed structural layout of the device, focusing on the viewing area of the grid array sensor. It illustrates the distribution of individual infrared detection elements, systematically arranged in a column-row format to enable comprehensive temperature mapping of the ear canal and tympanic membrane.

FIG. 5 details the interface of the grid array sensor, showing its spatial arrangement relative to the anatomical features of the ear, including the ear canal, positioning within the ear, and alignment with the tympanic membrane for optimal thermal detection.

FIG. 6 illustrates a fully exploded view of the ear infection detection device, revealing all major critical components and their assembly relationships within the unit.

FIG. 7 presents a detailed view of the grid array sensor, illustrating the precise distribution of individual infrared detection elements arranged in a structured column-row format. It also highlights the individualized pixel mapping locations, ensuring accurate positioning and enabling comprehensive thermal mapping for precise temperature analysis.

FIG. 8 provides a block diagram of the system architecture and electronics, outlining the major functional components, including the sensor interface, signal processing unit, wireless communication module, power management system, and user interface controls.

FIG. 9 illustrates an algorithmic flow diagram detailing the processing of temperature data collected by the grid array sensor. It outlines variations in temperature readings, graphical representation of thermal patterns, and the identification of potential signs of infection based on detected anomalies.

FIG. 10a-b presents an algorithmic flow diagram depicting the data collection and operational workflow of the device. It details processes including temperature mapping, differential calculations, anomaly detection, real-time data display, and automated reporting of diagnostic findings.

FIG. 11a-b provides an algorithmic flow diagram illustrating the sequence of operations involved in inserting the device into the ear canal. It includes steps for data collection, positional determination, focused analytics for enhanced accuracy, real-time alerts, continuous calibration for sensitivity adjustments, and the visualization of processed data for user interpretation.

FIG. 12a-b presents an algorithmic flow diagram outlining the complete process of inserting the device into the ear, capturing sensor data, and executing advanced data processing techniques. It highlights the integration of AI-driven machine learning algorithms for differential analysis, the creation of individualized thermal profiles, detection of infection indicators, and the secure storage and reporting of diagnostic results.

FIG. 13 is a flow diagram of an example process 1300 for the generation and training of artificial intelligence models (also referred to herein as machine learning models) to perform one or more of the processes described herein, according to an example described herein.

### DETAILED DESCRIPTION

The Infrared Grid Array Ear Infection Detection Device disclosed herein represents a revolutionary advancement in diagnosing otitis media and related ear infections. By integrating a spatially distributed grid array infrared sensor with an advanced microprocessor, this device not only overcomes the limitations of traditional detection methods but also reduces human error by eliminating the subjectivity inherent in manual visual interpretations. For example, a distributed sensor approach can reduce misinterpretations by up to 40% compared to single-point methods. In addition, the robust design described herein is supported by extensive research and rigorous testing, ensuring that even subtle temperature changes associated with early infection stages are accurately detected. The grid array configuration may be engineered to maximize both sensitivity and resolution, providing a reliable tool that may be poised to transform standard diagnostic practices. This innovative approach marks a significant leap forward in the field of medical diagnostics, offering clinicians a powerful, objective tool to improve patient outcomes and reduce the incidence of misdiagnoses. Overall, the device's design offers enhanced accuracy, ease of use, and adaptability to various environmental conditions, ensuring its effectiveness across a wide range of clinical scenarios.

Generally, ear infection detection has relied on visual inspection using an otoscope or indirect diagnostic methods such as tympanometry and acoustic reflectometry. These traditional methods are highly dependent on practitioner skill and experience, often leading to errors from subjective interpretations. For instance, two clinicians might differ on whether a mildly reddened eardrum indicates early inflammation, resulting in inconsistent diagnoses. In addition, methods like tympanometry may be unreliable in young children due to anatomical variations that hinder accurate readings. Conventional thermography has similarly struggled with issues such as positional inaccuracies and environmental interferences, leading to a high risk of diagnostic mistakes—particularly when using single-point infrared thermometers that require precise alignment. The inherent variability of these methods can result in significant delays in diagnosis, improper treatment plans, and increased patient discomfort, thereby emphasizing the need for a more reliable and objective diagnostic solution.

The device described herein may have a grid array infrared sensor that may ensure comprehensive coverage of the ear canal, eliminating the need for meticulous positioning that has challenged previous diagnostic tools. Individual sensor elements are strategically placed to detect infection indicators across the ear canal, thereby reducing errors caused by misalignment or patient movement. This distributed approach is analogous to industrial sensor arrays used in quality control, where multiple detection points have been proven to enhance overall accuracy and reliability. By capturing high-resolution thermal maps, the device is capable of detecting subtle temperature variations across the eardrum and surrounding tissues—facilitating the early detection of acute otitis media and reducing reliance on sometimes ambiguous visual cues. This dynamic, three-dimensional mapping of temperature gradients provides clinicians with detailed, actionable data that supports early intervention and more precise treatment planning.

An integrated microprocessor may process infection data in real time using advanced algorithms that compensate for variations in sensor positioning and environmental factors. By incorporating machine learning techniques trained on extensive clinical datasets, the device can accurately distinguish between normal thermal variations and clinically significant temperature spikes. This intelligent processing not only improves diagnostic precision but also reduces the incidence of false positives often observed in visual examinations—such as mistaking a slight temperature rise due to ambient heat for an infection. Moreover, the advanced algorithms can differentiate between inflammation and fluid buildup by directly analyzing thermal gradients, providing a clear advantage over tympanometry, which often yields inconclusive results in cases of narrow pediatric ear canals. The system's ability to adapt to a wide range of operating conditions is a testament to the robust design of its software architecture, which continuously learns and refines its predictive capabilities over time.

Designed with user comfort and safety in mind, the device can feature a non-intrusive design that passively captures thermal data without requiring probe insertion or pressurization of the ear canal. This is especially beneficial in pediatric care, where minimizing patient discomfort and anxiety is paramount to obtaining accurate diagnostic results. Traditional methods that involve physical insertion can cause significant distress in young patients, potentially leading to incomplete examinations or the need for repeat tests. The non-invasive nature of the grid array device not only streamlines the diagnostic process but also increases the likelihood of collecting high-quality data on the first attempt. Its ergonomic design minimizes physical contact, thereby reducing the risk of secondary infections and ensuring a more comfortable experience for patients while simplifying the procedure for healthcare providers.

A built-in display unit may provide real-time, quantified infection detection data with color-coded thermal maps that are easier to interpret than traditional grayscale or subjective visual outputs. This digital interface may significantly reduce the potential for human error by converting complex thermal data into clear, intuitive visual representations. In contrast to conventional otoscopes—which require experienced clinicians to interpret subtle visual cues—the display presents objective, standardized data that clinicians can use to make rapid, informed decisions. The clarity and precision of the digital display are comparable to modern digital radiography systems, where consistent, high-quality image outputs have minimized interpretive discrepancies among radiologists. Additionally, interactive features allow clinicians to zoom in on specific areas and compare current readings with historical data, further enhancing the diagnostic process.

Furthermore, wireless connectivity may enable seamless integration into modern healthcare systems, facilitating remote monitoring and efficient data collection. This feature is particularly advantageous in settings such as intensive care units or home healthcare environments, where continuous patient monitoring is essential. Unlike older, cumbersome wired systems, the wireless design allows the device to communicate effortlessly with electronic health records (EHR) systems, ensuring that patient data is updated in real time. In telemedicine applications, the remote transmission of accurate, high-resolution thermal data enables specialists to provide prompt consultations and diagnoses without requiring in-person visits. This capability not only expedites the treatment process but also helps extend advanced diagnostic services to remote or underserved areas, effectively bridging the gap between patients and high-quality medical care.

The device is also engineered to adapt to varying ambient conditions through built-in calibration protocols and adaptive signal processing techniques that ensure consistent performance. This dynamic adjustment mitigates issues such as temperature drift and sensor instability—challenges that have historically plagued conventional infrared devices. For example, in emergency rooms or field hospitals where environmental conditions can fluctuate rapidly, the sensor's adaptive calibration ensures that reading remains both accurate and reliable. This robustness in variable settings is a significant improvement over systems that do not compensate for rapid ambient changes. The adaptive technology may also continuously monitor external conditions and automatically adjust the sensor output, ensuring that the diagnostic data remains precise regardless of environmental variability.

Beyond overcoming visual obstructions such as earwax—which can distort light-based detection methods—the device is particularly adept at detecting fever-related acute otitis media. By measuring temperature at multiple points simultaneously, the disclosed device can identify early signs of infection long before visible symptoms, such as redness or swelling, appear. This multi-point detection strategy provides a clear advantage over other methods that often miss early-stage infections. The ability to generate a comprehensive thermal profile of the ear allows clinicians to pinpoint even small anomalies indicative of infection.

As such, the Infrared Grid Array Ear Infection Detection Device described herein sets a new standard in medical diagnostics by addressing critical challenges such as positional inaccuracies, subjective visual interpretation, and patient discomfort. Its innovative combination of grid array sensor technology, non-intrusive design, real-time data processing, and wireless connectivity not only enhances diagnostic accuracy but also elevates overall patient care. By refining and surpassing the limitations of historical diagnostic technologies, this device paves the way for more reliable and early detection of ear infections. Its comprehensive, objective approach contributes significantly to the advancement of modern medical care, ensuring that clinicians can deliver more effective, timely, and personalized treatment to their patients.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated or described in connection with one embodiment may be combined with the features of other embodiments, including as between systems and methods. Such modifications and variations are intended to be included within the scope of the appended claims.

Additional details are described below with reference to several example embodiments.

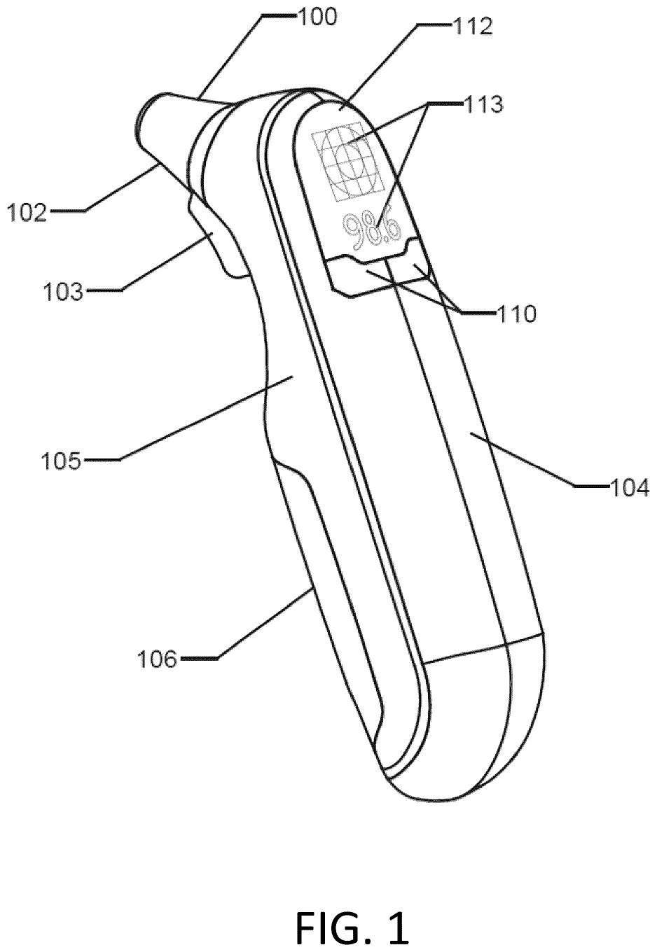

FIG. 1 illustrates an external isometric view of the ear infection detection device, showcasing its key components, including the sensor array, housing structure, and user interface elements. Prominently featured is the Speculum (100), which may guide the device securely into the ear canal, while the Lens (102) may focus light onto the sensor array to ensure clear imaging. The Ejector (103) is visible as a mechanism for replacing disposable components, and the overall structure is encapsulated by the Top Housing (104) and Bottom Housing (105), which provide protection and ergonomic design. The Battery Door (106) may offer convenient access for power management, and the device's operational functions may be controlled via Switches (110) that manage power, measurement, and timing. Enhancing the user interface, the Display Cover (112) may protect the screen, and the indicator elements (113), including an LCD, touch screen, and LEDs, may deliver real-time diagnostic feedback and information.

As such, FIG. 1 illustrates an external isometric view of the ear infection detection device, showcasing its overall design and key components. The device features a compact, handheld form factor with a streamlined shape suitable for medical examination use. The main body of the device comprises the Top Housing (104) and Bottom Housing (105) that encase the internal components and provide an ergonomic grip for the user. At the front end of the device, a Speculum (100) extends outward, designed to interface with the patient's ear canal during examination.

The user interface elements of the device are prominently displayed on the upper surface. A series of Switches (110) may allow for control of various device functions, potentially including power, measurement initiation, and timing operations. Adjacent to the Switches (110), a Display Cover (112) may protect an underlying screen or display panel. The indicator elements (113) may include an LCD, touch screen, or LED indicators, providing real-time feedback and diagnostic information to the healthcare professional using the device.

The optical and sensing components of the device may be concentrated at the device's front end. A Lens (102) may be positioned behind the Speculum (100), focusing incoming light and/or infrared radiation onto the internal sensor array. This configuration may allow for precise measurement of tympanic temperatures across multiple points within the ear canal. An Ejector (103) mechanism is visible near the front of the device, which may facilitate the hygienic replacement of disposable components such as speculum covers between patient examinations.

The device's power management and maintenance features may be integrated into its design. A Battery Door (106) is located on the lower portion of the housing, providing convenient access for battery replacement or recharging. This design element suggests that the device may be cordless and portable, enhancing its usability in various clinical settings. The overall construction of the device, with its sealed housing and minimal external openings, may contribute to ease of cleaning and durability in medical environments where maintaining sterility is crucial.

It should be noted that the exchange of data and/or information as described herein may be performed only in situations where a user has provided consent for the exchange of such information. For example, a user may be provided with the opportunity to opt in and/or opt out of data exchanges between devices and/or with the systems and/or for performance of the functionalities described herein. Additionally, when one of the devices is associated with a first user account and another of the devices is associated with a second user account, user consent may be obtained before performing some, any, or all of the operations and/or processes described herein.

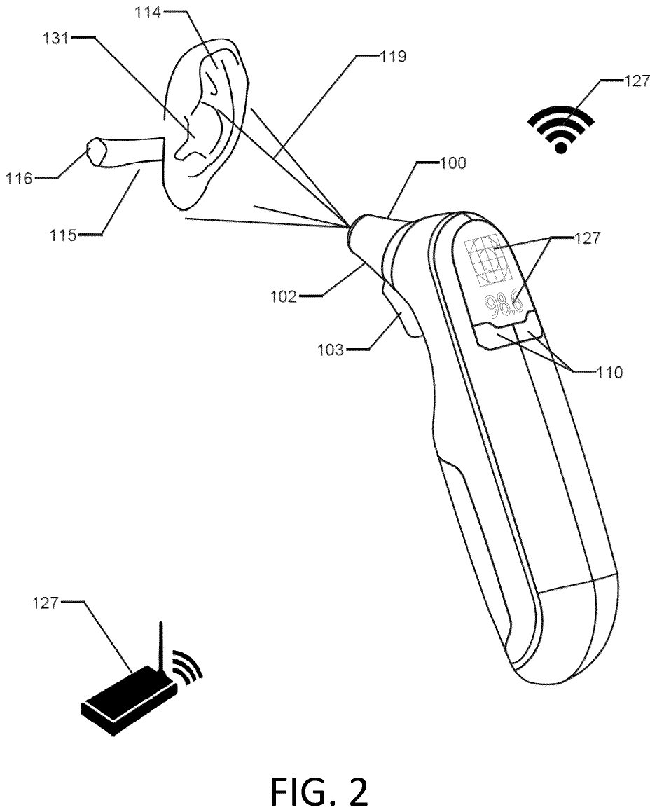

FIG. 2 provides a perspective view of the ear infection detection device, highlighting its interaction with the tympanic membrane (116) and surrounding anatomical structures. The Speculum (100) is designed to facilitate proper alignment within the ear canal (115), ensuring an unobstructed path for the Lens (102) to capture thermal data accurately. The Ejector (103) enables easy removal or replacement of disposable components after various uses. Various Switches (110) allow users to control device functions, such as power, measurement activation, and data storage. The device is adaptable for use in the Right and/or Left Ear (114) across different patient age groups, ensuring versatility in both pediatric and adult applications. Its Field of View and Position Accuracy (119) are optimized to maintain consistent reading despite minor patient movements. The user interface displays real-time diagnostic data using clear indicators, while Data Transfer (127) capabilities-via Wi-Fi, Bluetooth, or cellular connectivity-enable seamless integration with electronic health records and telemedicine platforms. Additionally, the device accounts for potential Occlusion (131) from earwax or foreign objects, incorporating algorithms to adjust readings and maintain diagnostic accuracy.

As such, The device features the Speculum (100) at its front end, designed to interface with the ear canal (115) and guide the device into proper position. Behind the Speculum, a Lens (102) is positioned to focus incoming infrared radiation onto the internal sensor array. The device's main body houses various control Switches (110) for managing device functions. The illustration also depicts the tympanic membrane (116), which is the primary target for temperature measurement and infection detection.

FIG. 2 further demonstrates the device's adaptability for use in both the Right and Left Ear (114) across different patient age groups, from pediatric to adult applications. This versatility is achieved through the device's Field of View and Position Accuracy (119), which may be optimized to maintain consistent readings despite minor patient movements or anatomical variations. The Ejector (103) mechanism is also visible, may allow for easy removal or replacement of disposable components such as speculum covers, enhancing the device's hygiene and reusability in clinical settings.

FIG. 2 further highlights the device's advanced connectivity features, represented by the Data Transfer (127) capabilities. These may include Wi-Fi, Bluetooth, or cellular connectivity options, enabling seamless integration with electronic health records and telemedicine platforms. This wireless functionality may allow for real-time data transmission, remote monitoring, and efficient integration into modern healthcare systems. The user interface may display real-time diagnostic data using clear indicators, providing immediate feedback to healthcare professionals during examinations.

FIG. 2 also addresses a common challenge in ear examinations-the presence of Occlusion (131) from earwax or foreign objects. The device may be designed to account for such obstructions, incorporating algorithms to adjust readings and maintain diagnostic accuracy even in less-than-ideal examination conditions. This feature, combined with the device's optimized field of view and position accuracy 119, contributes to its ability to provide reliable and consistent measurements across a wide range of patient scenarios, potentially improving the overall efficacy of ear infection diagnoses in clinical practice.

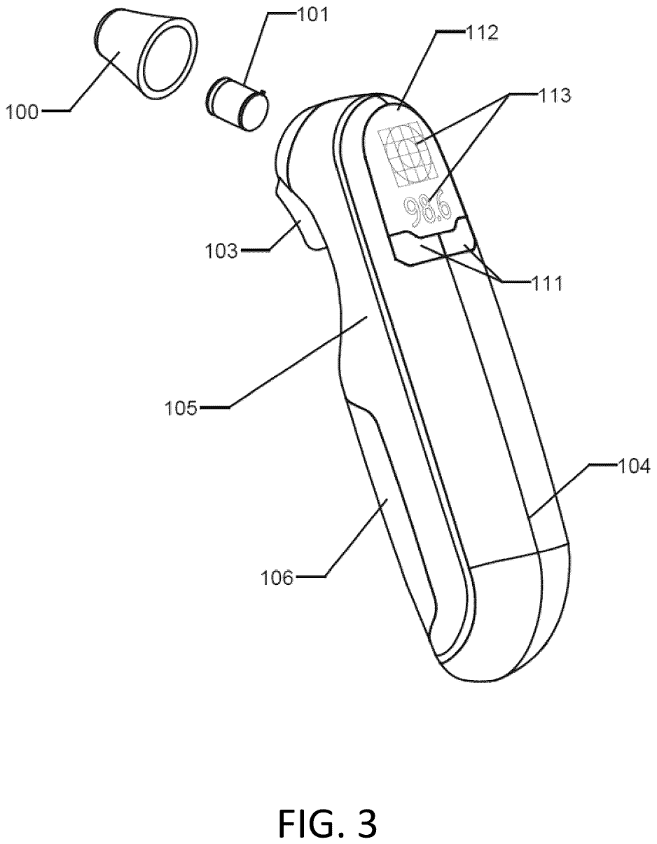

FIG. 3 presents an isometric exploded view of the ear infection detection device, showcasing its key structural and functional components. The Speculum (100) is designed to encase and protect the Grid Sensor (101), which measures voltages, ambient temperature, and thermal variations within the ear canal. The Display Cover (112) shields the indicator system (113), which may include an LCD, touch screen, or LEDs for clear diagnostic visualization. The User Interface (111), incorporating a buzzer and haptic feedback, ensures intuitive operation and alerts users to measurement results. The Top Housing (104) and Bottom Housing (105) form the main structural enclosure, securing all internal components. The Battery Door (106) allows for easy access to the power supply, ensuring uninterrupted functionality. Lastly, the Ejector (103) facilitates quick removal and replacement of the speculum, maintaining hygiene and device longevity.

The Speculum (100) is prominently featured at the front of the device, designed to interface with the patient's ear canal during examination. Behind the Speculum, the Grid Sensor (101) is positioned, which may be responsible for measuring voltages, ambient temperature, and/or thermal variations within the ear canal. The device's main body is formed by the Top Housing (104) and Bottom Housing (105), which encase and protect the internal components. Various user interface elements are visible, including the Display Cover (112) and the User Interface (111) components.

The exploded view in FIG. 3 illustrates the spatial relationships and assembly order of the device's components. The Speculum (100) may be designed to securely attach to the front of the device, encasing and protecting the Grid Sensor (101). The Top Housing (104) and Bottom Housing (105) may be configured to fit together, forming a sealed enclosure that houses the internal electronics and provides an ergonomic grip for the user. The Battery Door (106) may be integrated into the housing, allowing for easy access to the power supply without compromising the device's structural integrity.

FIG. 3 also highlights several unique features of the ear infection detection device. The Grid Sensor (101) may represent an advanced sensing technology capable of capturing detailed thermal data across multiple points in the ear canal. The User Interface (111), which may incorporate a buzzer and haptic feedback, may provide an intuitive means of operation and alert users to measurement results. The Display Cover (112) may protect an underlying indicator system (113), which may include an LCD, touch screen, or LEDs for clear diagnostic visualization. These features may contribute to the device's ability to provide accurate, real-time diagnostic information in a user-friendly manner.

The components shown in FIG. 3 may work together to achieve the intended functionality of the ear infection detection device. The Speculum (100) may guide the Grid Sensor (101) into the proper position within the ear canal, allowing for precise measurement of thermal variations. The sensor data may be processed by internal electronics housed within the Top Housing (104) and Bottom Housing (105). The results may then be displayed through the indicator system (113) protected by the Display Cover (112), providing immediate feedback to the healthcare professional. The Ejector (103) may facilitate quick removal and replacement of the Speculum, maintaining hygiene between examinations. This integrated design may allow for efficient, accurate, and hygienic detection of ear infections, addressing the limitations of traditional diagnostic methods.

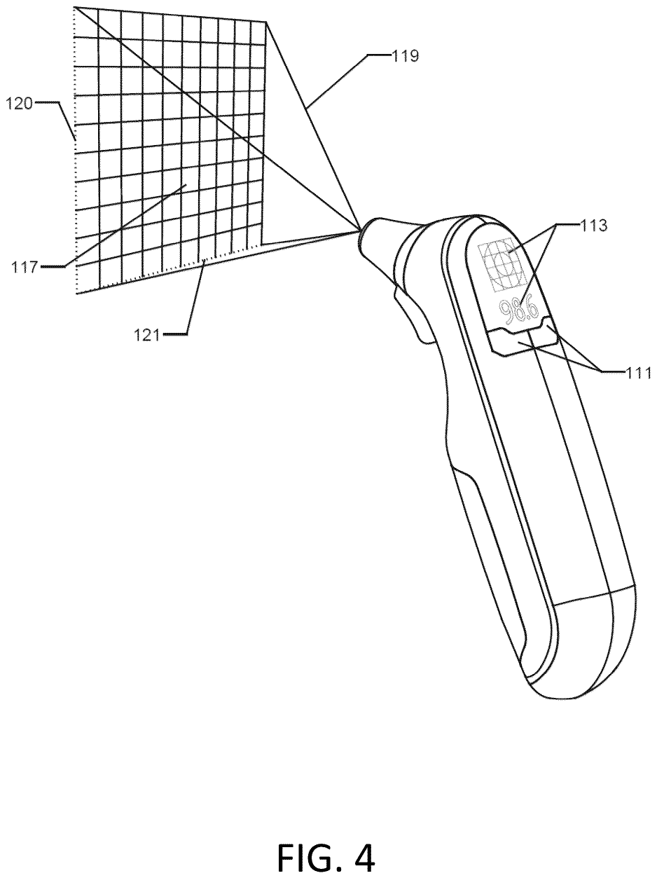

FIG. 4 provides a detailed structural layout of the ear infection detection device, emphasizing the viewing area of the Grid Array (117). This array consists of multiple infrared detection elements systematically arranged along the Grid X Axis (120) and Grid Y Axis (121), forming a precise column-row format for capturing thermal data. The Field of View and Position Accuracy (119) ensures that the sensor elements align correctly with the ear canal and tympanic membrane for accurate temperature mapping. The User Interface (111), which includes a buzzer and haptic feedback, enhances usability by providing real-time alerts. Additionally, the Indicator system (113), featuring an LCD, touch screen, or LED display, visually presents diagnostic results, ensuring clear interpretation of the collected thermal data.

As such, FIG. 4 illustrates a systematic arrangement of infrared detection elements organized along a Grid X Axis (120) and Grid Y Axis (121). This grid structure forms the core sensing component of the device, enabling comprehensive thermal data capture across multiple points within the ear canal. The overall layout demonstrates how the device integrates advanced sensing technology with user interface elements to create a cohesive diagnostic tool.

The Grid Array (117) is depicted as a matrix of individual sensing elements, each capable of detecting infrared radiation emitted from the tympanic membrane and surrounding tissues. The precise column-row format of the array may allow for high-resolution thermal mapping of the ear canal. This arrangement may enable the device to capture detailed temperature variations across different regions of the examined area, potentially improving the accuracy of infection detection compared to single-point measurement methods.

The Field of View and Position Accuracy (119) component shown in FIG. 4 may ensure reliable measurements. This feature may help maintain proper alignment between the sensor elements and the anatomical structures of the ear, even with minor patient movements during examination. By optimizing the field of view and position accuracy, the device may be able to provide consistent and dependable readings across various patient scenarios, potentially enhancing its clinical utility in diverse healthcare settings.

FIG. 4 also highlights the user interface components of the device. The User Interface (111), which may incorporate haptic feedback and auditory alerts through a buzzer, is shown as an integral part of the device's design. Additionally, the Indicator system (113) is depicted, which may include visual display elements such as an LCD, touch screen, or LED indicators. These interface components may work in concert to provide healthcare professionals with clear, real-time diagnostic information and alerts, potentially streamlining the examination process and facilitating rapid, informed decision-making in clinical practice.

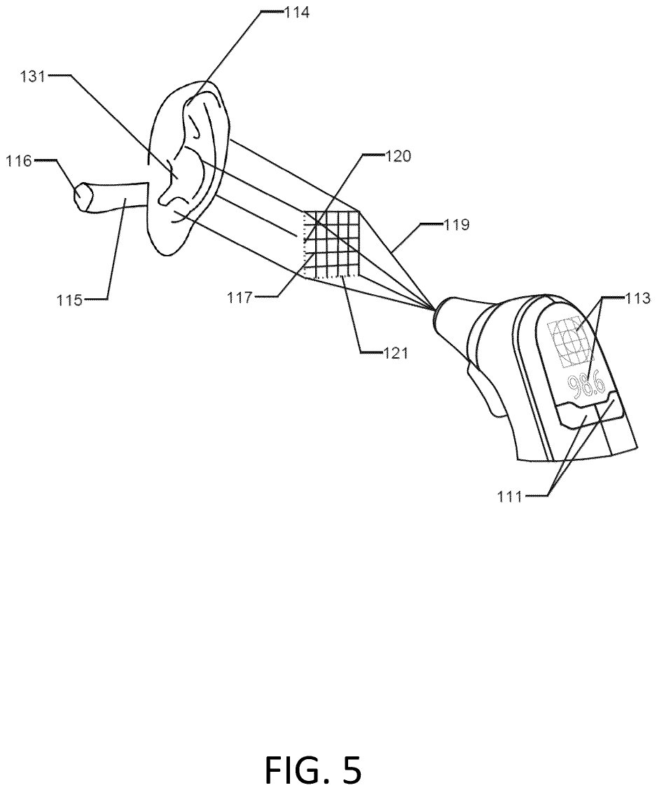

FIG. 5 illustrates the interface of the Grid Array (117) sensor, showcasing its spatial arrangement relative to key anatomical structures of the ear. The sensor is precisely aligned along the Grid X Axis (120) and Grid Y Axis (121) to ensure comprehensive thermal detection within the Ear Canal (115) and accurate temperature mapping of the Tympanic Membrane (116). The Field of View and Position Accuracy (119) are optimized to maintain proper sensor alignment for reliable diagnostics. The User Interface (111), featuring a buzzer and haptic feedback, enhances usability by providing real-time alerts. Diagnostic results are displayed through the indicator system (113), which includes an LCD, touch screen, or LED display for clear interpretation. The device is designed for use across different age groups, accommodating Right and/or Left Ear detection in Adults, Infants, and Adolescents (114). Additionally, the system accounts for potential Occlusion issues (131), such as earwax or foreign objects, ensuring consistent and accurate readings.

The Grid Array (117) sensor is shown positioned within the Ear Canal (115), aligned to capture thermal data from the Tympanic Membrane (116). The device's structure is designed to accommodate examinations of both the Right and Left Ear (114) across various age groups, including adults, infants, and adolescents. This versatile design may allow for widespread applicability in diverse clinical settings.

The sensor's Grid X Axis (120) and Grid Y Axis (121) are depicted as forming a precise matrix, enabling detailed thermal mapping of the ear canal and tympanic membrane. This grid structure may allow for high-resolution temperature measurements across multiple points simultaneously. The Field of View and Position Accuracy (119) component is illustrated, suggesting the device's capability to maintain proper alignment and consistent readings even with slight patient movements or anatomical variations.

The User Interface (111) elements are shown integrated into the device's design, including features such as a buzzer and haptic feedback. These components may enhance the device's usability by providing immediate tactile and auditory alerts to the healthcare professional during the examination process. The indicator system (113) is also depicted, which may include visual display options such as an LCD, touch screen, or LED indicators, potentially offering clear and intuitive presentation of diagnostic results.

FIG. 5 also addresses potential challenges in ear examinations, such as Occlusion issues (131) that may arise from the presence of earwax or foreign objects. The illustration suggests that the device's design and algorithms may account for these obstructions, potentially maintaining measurement accuracy in less-than-ideal examination conditions. This feature, combined with the device's optimized sensor arrangement and interface design, may contribute to its ability to provide reliable and consistent measurements across a wide range of patient scenarios and clinical environments.

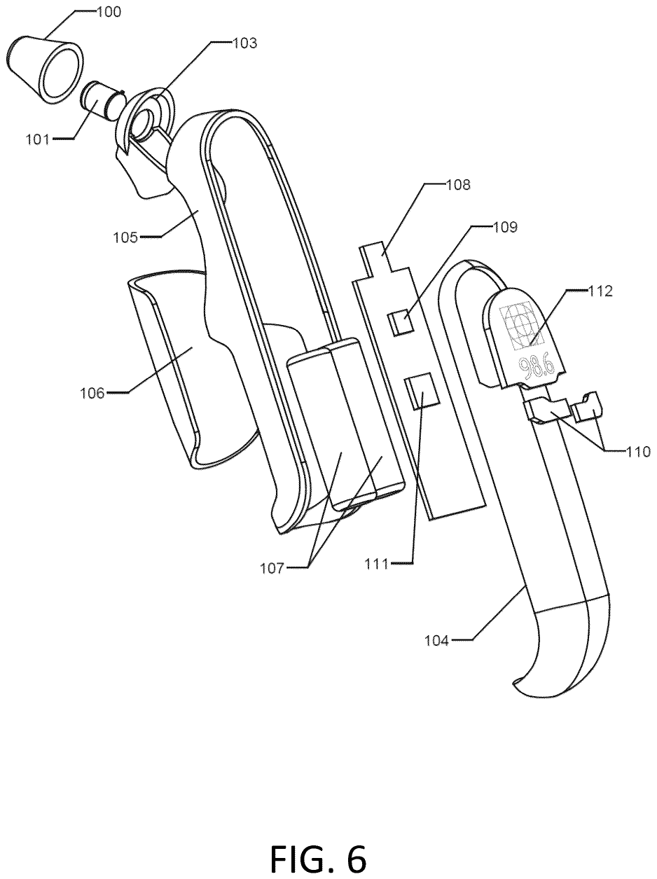

FIG. 6 provides a fully exploded view of the ear infection detection device, revealing the critical components and their assembly relationships. The Speculum (100) is positioned at the front to guide the sensor alignment and ensure proper placement within the ear. The Grid Sensor (101) is housed securely within the assembly, enabling precise voltage, ambient, and temperature measurements. The Ejector (103) mechanism facilitates the removal and replacement of disposable specula. The structural integrity of the device is maintained by the Top Housing (104) and Bottom Housing (105), which encase and protect the internal components. The Battery Door (106) provides access to the Batteries and Power Supply (107), ensuring reliable operation. The Printed Circuit Board Assembly (108) integrates the Microprocessor(s) (109), responsible for processing sensor data and executing device functions. User controls, including the Switches (110) for power, measurement, memory timer, and unit selection (Celsius/Fahrenheit), are strategically placed for ease of use. The User Interface (111), incorporating a buzzer and haptic feedback, provides immediate alerts and guidance. Finally, the Display Cover (112) protects the visual interface, ensuring durability while allowing clear visibility of diagnostic results.

The Speculum (100) is positioned at the front of the device, designed to interface with the patient's ear canal and guide the sensor alignment. Behind it, the Grid Sensor (101) is securely housed, enabling precise measurements of voltage, ambient temperature, and thermal variations within the ear canal. The overall structure of the device may be formed by the Top Housing (104) and Bottom Housing (105), which encase and protect the internal components while providing an ergonomic form factor for handheld use.

The user interface elements of the device are prominently displayed in the exploded view. The Switches (110) for power, measurement, memory timer, and unit selection (Celsius/Fahrenheit) are strategically placed for easy access and operation. The User Interface (111) incorporates a buzzer and haptic feedback system, potentially enhancing the device's usability by providing immediate tactile and auditory alerts to the healthcare professional during examinations. The Display Cover (112) protects the visual interface, which may include an LCD, touch screen, or LED indicators for clear presentation of diagnostic results.

Internal components crucial to the device's functionality are revealed in the exploded view. The Printed Circuit Board Assembly (108) integrates the Microprocessor(s) (109), which may be responsible for processing sensor data and executing various device functions. This assembly may form the core of the device's computational capabilities, potentially enabling advanced data analysis and real-time diagnostics. The Battery Door (106) provides access to the Batteries and Power Supply (107), ensuring a reliable and easily maintainable power source for the device's operation.

Several auxiliary components contribute to the device's overall functionality and ease of use. The Ejector (103) mechanism may facilitate the quick removal and replacement of disposable specula, potentially enhancing the device's hygiene management between patient examinations. This feature, combined with the device's modular design, may allow for efficient maintenance and preparation of the device in clinical settings. The exploded view also illustrates how these various components are designed to fit together, potentially providing insights into the device's assembly process and the interdependencies between its different functional elements.

As used herein, a processor may include multiple processors and/or a processor having multiple cores. Further, the processors may comprise one or more cores of different types. For example, the processors may include application processor units, graphic processing units, and so forth. In one implementation, the processor may comprise a microcontroller and/or a microprocessor. The processor(s) 108 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that can be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) may possess its own local memory, which also may store program components, program data, and/or one or more operating systems.

The memory may include volatile and nonvolatile memory, removable and non-removable media implemented in any method or technology for storage of information, such as computer-readable instructions, data structures, program component, or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory may be implemented as computer-readable storage media (“CRSM”), which may be any available physical media accessible by the processor(s) 108 to execute instructions stored on the memory. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the processor(s).

Further, functional components may be stored in the respective memories, or the same functionality may alternatively be implemented in hardware, firmware, application specific integrated circuits, field programmable gate arrays, or as a system on a chip (SoC). In addition, while not illustrated, each respective memory, such as memory discussed herein may include at least one operating system (OS) component that is configured to manage hardware resource devices such as the network interface(s), the I/O devices of the respective apparatuses, and so forth, and provide various services to applications or components executing on the processors. Such OS component may implement a variant of the FreeBSD operating system as promulgated by the FreeBSD Project; other UNIX or UNIX-like variants; a variation of the Linux operating system as promulgated by Linus Torvalds; the FireOS operating system from Amazon. com Inc. of Seattle, Washington, USA; the Windows operating system from Microsoft Corporation of Redmond, Washington, USA; LynxOS as promulgated by Lynx Software Technologies, Inc. of San Jose, California; Operating System Embedded (Enea OSE) as promulgated by ENEA AB of Sweden; and so forth.

The network interface(s) may enable messages between the components and/or devices shown in environment 100 and/or with one or more other remote systems, as well as other networked devices. Such network interface(s) may include one or more network interface controllers (NICs) or other types of transceiver devices to send and receive messages over the network.

For instance, each of the network interface(s) may include a personal area network (PAN) component to enable messages over one or more short-range wireless message channels. For instance, the PAN component may enable messages compliant with at least one of the following standards IEEE 802.15.4 (ZigBee), IEEE 802.15.1 (Bluetooth), IEEE 802.11 (WiFi), or any other PAN message protocol. Furthermore, each of the network interface(s) 110 may include a wide area network (WAN) component to enable message over a wide area network.

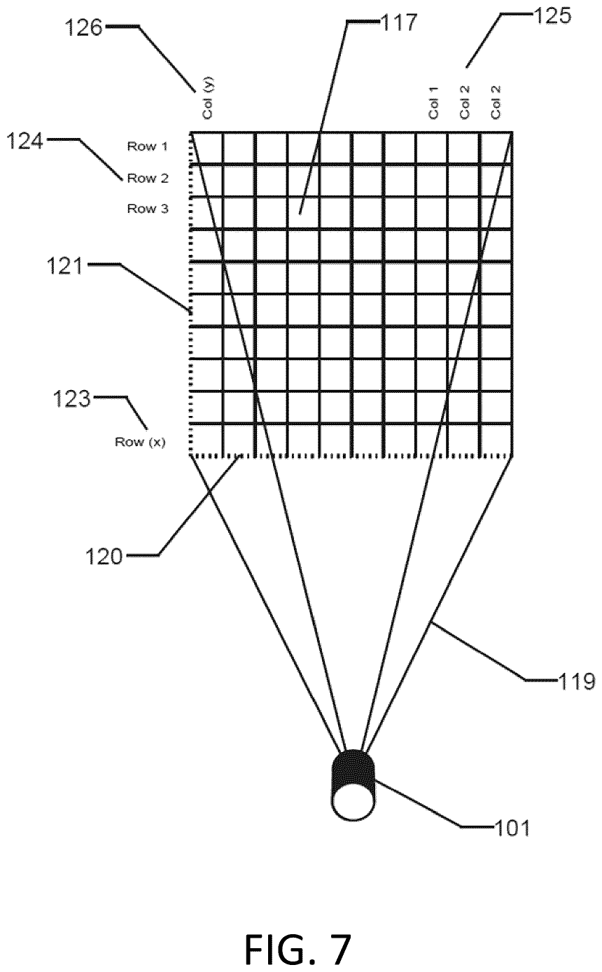

FIG. 7 presents a detailed view of the Grid Sensor (101), showcasing the structured arrangement of individual infrared detection elements essential for precise thermal mapping. The Field of View and Position Accuracy (119) ensures optimal sensor alignment for accurate readings. The Grid X Axis (120) and Grid Y Axis (121) define the spatial organization of the detection elements, systematically arranged in a column-row format. The X Axis Row Count (123) and Y Axis Row Count (125) determine the number of detection rows, while the X Axis Row Number (124) and Y Axis Row Number (126) specify the exact positioning of each infrared detection element within the grid. The Grid Array (117) enables individualized pixel mapping, ensuring comprehensive temperature analysis by capturing precise thermal data from various locations within the ear canal.

The Grid Sensor (101) is depicted as a rectangular array with multiple rows and columns, each representing a distinct sensing point. This grid-like structure enables comprehensive thermal mapping of the target area, potentially allowing for high-resolution temperature measurements across the ear canal and tympanic membrane.

As such in FIG. 7, FIG. 7 illustrates the spatial organization of the detection elements through the Grid X Axis (120) and Grid Y Axis (121). These axes define the column-row format of the sensor array, providing a systematic framework for positioning each infrared detection element. The X Axis Row Count (123) and Y Axis Row Count (125) are indicated, specifying the number of rows in each direction and thus determining the overall resolution and coverage area of the sensor.

Within the grid structure, individual sensing elements may be precisely positioned using the X Axis Row Number (124) and Y Axis Row Number (126). These coordinates may allow for accurate localization of temperature readings, potentially enabling the device to create detailed thermal maps of the examined area. The Grid Array (117) is shown as the collective arrangement of these individual elements, forming a cohesive sensing unit capable of capturing thermal data from multiple points simultaneously.

The Field of View and Position Accuracy (119) component is depicted in relation to the grid sensor, suggesting the device's capability to maintain optimal alignment and accuracy during measurements. This feature may contribute to the sensor's ability to provide consistent and reliable readings across various examination scenarios, potentially enhancing the overall diagnostic capabilities of the ear infection detection device.

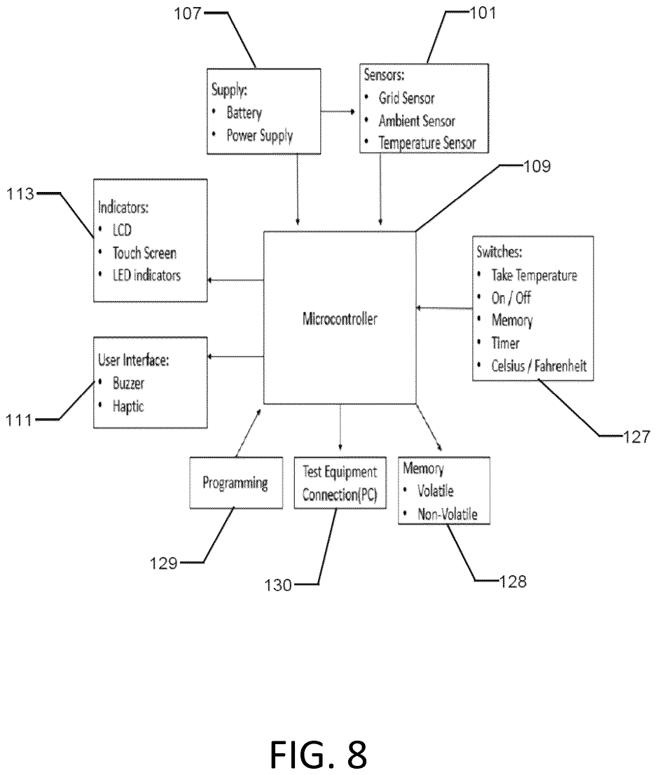

FIG. 8 provides a block diagram of the system architecture and electronics, detailing the interaction between key functional components. The Batteries and Power Supply (107) manage energy distribution to all system elements, ensuring stable operation. The Grid Sensor (101) captures infrared data and sends signals to the Microprocessor(s) (109), which processes the information for accurate temperature analysis. Wireless communication is facilitated through Data Transfer, Wi-Fi, Bluetooth, and Cellular modules (127), enabling seamless connectivity for remote monitoring and data storage. Memory, including Volatile and Non-Volatile (128) storage, retains diagnostic data and system configurations. Programming and Calibration (129) ensure proper device functionality, while Calculations, Sensitivity, Test, Error Reporting, and Malfunctions (130) manage performance accuracy and system diagnostics. The User Interface, Buzzer, and Haptic feedback (111) provide real-time alerts, and the Indicator, LCD, Touch Screen, and LEDs (113) display diagnostic results and system status.

At the center of FIG. 8 is the Microcontroller unit, which serves as the primary processing hub for the system. This central component interfaces with various peripheral modules and sensors, coordinating the device's overall functionality and data processing capabilities. The power management system is depicted in the diagram, comprising the Batteries and Power Supply (107) components. This subsystem may be responsible for providing and regulating the electrical power required by all other components of the device. The power supply's integration with the microcontroller suggests a managed approach to energy distribution, potentially allowing for power optimization and extended operational life of the device.

The sensing capabilities of the device are represented by multiple components in the diagram. The Grid Sensor (101) is shown as a key input device, likely responsible for capturing the infrared thermal data from the ear canal. Additional sensing elements include the Ambient Sensor and Temperature Sensor, which may provide contextual environmental data to enhance the accuracy of the primary measurements. These sensors interface directly with the microcontroller, allowing for real-time data acquisition and processing.

The user interface and output systems are detailed in the lower portion of the diagram. These include the LCD, Touch Screen, and LED indicators (113), which may provide visual feedback and interactive control for the user. The User Interface block (111) incorporates Buzzer and Haptic feedback elements, suggesting a multi-modal approach to user interaction. Various switches (110) for functions such as temperature measurement, power control, and unit selection are also depicted, illustrating the device's operational controls. This comprehensive interface design may enable intuitive operation and clear presentation of diagnostic results to healthcare professionals.

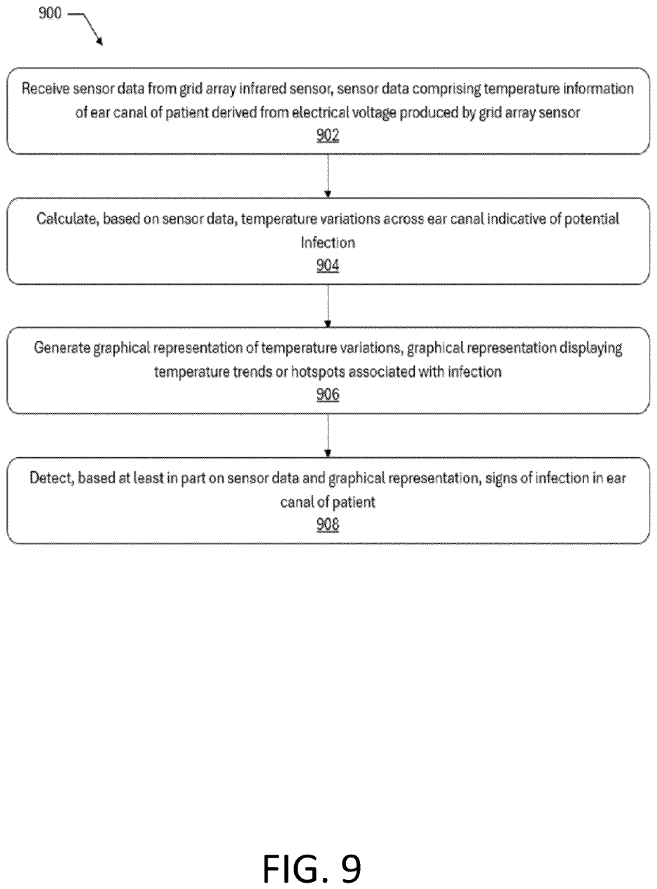

FIGS. 9-13 illustrate processes associated with a novel grideye sensor. The processes described herein are illustrated as collections of blocks in logical flow diagrams, which represent a sequence of operations, some or all of which may be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the process, or alternative processes, and not all of the blocks need be executed. For discussion purposes, the processes are described with reference to the environments, architectures and systems described in the examples herein, such as, for example those described with respect to FIGS. 1-8, although the processes may be implemented in a wide variety of other environments, architectures and systems.

FIG. 9 illustrates an algorithmic flow diagram that outlines the sequential processing of temperature data collected by the grid array sensor. The order in which the operations or steps are described is not intended to be construed as a limitation, and any number of the described operations may be combined in any order and/or in parallel to implement process 900.

At block 902, the process 900 includes receiving sensor data, where infrared readings from the grid array are gathered and pre-processed for accuracy. This step may involve collecting raw temperature data from multiple points across the ear canal and tympanic membrane using the grid sensor array. The sensor data may be digitized and formatted for further processing. This initial data acquisition stage may interface with the Grid Sensor (101) and Microprocessor(s) (109) components described above to ensure proper data capture and preliminary filtering of noise or artifacts.

At block 904, the process 900 may include analyzing temperature variations across different regions of the ear canal and tympanic membrane. This step may involve complex algorithmic processing of the raw sensor data to identify temperature patterns and anomalies. The calculations may include statistical analysis, spatial mapping, and comparison to predefined temperature thresholds indicative of infection. This stage may heavily utilize the computational capabilities of the Microprocessor(s) (109) and may reference data stored in the Memory (128) for comparative analysis against known infection patterns.

At block 906, the process 900 may include generating graphics that visually represent thermal patterns, providing a clear indication of temperature distribution. This step may involve creating color-coded heat maps, 3D temperature models, or other visual representations of the ear canal's thermal profile. The graphics generation process may interface with the display components such as the LCD or Touch Screen (113) to render these visualizations. This stage may also involve data compression or optimization techniques to efficiently represent complex thermal data in an easily interpretable format for healthcare professionals.

At block 908, the process 900 may include evaluating the graphical patterns to identify potential anomalies, such as localized temperature spikes, which may indicate signs of infection. This step may involve advanced pattern recognition algorithms, potentially utilizing machine learning techniques, to analyze the generated graphics and identify characteristics associated with ear infections. The detection process may interface with the User Interface (111) components to provide immediate feedback, such as alerts or diagnostic suggestions, based on the analysis results. This stage may also trigger data storage operations to record the diagnostic findings in the device's Memory (128) for future reference or transmission via the Data Transfer modules (127).

The process 900 may also include any or all of the following operations and/or details.

The acute otitis media ear infection detection device may incorporate a grid array infrared sensor that generates sensor data by producing electrical voltages corresponding to detected infrared radiation in the ear canal. These voltages are used to calculate temperature values for infection detection. The device's processors receive this sensor data, which comprises temperature information derived from the electrical voltages. The processors then calculate temperature variations across the ear canal that may indicate potential infection. A graphical representation of these temperature variations is generated, displaying temperature trends or hotspots associated with infection. By analyzing both the sensor data and the graphical representation, the device detects signs of infection in the patient's ear canal. This integrated approach may allow for comprehensive and visual assessment of ear canal temperatures, potentially enhancing the accuracy and interpretability of infection detection.

The device's grid array infrared sensor may incorporate multiple individual sensors arranged in a spatially distributed grid pattern. This configuration may allow for comprehensive coverage of the ear canal, potentially capturing thermal data from various angles and depths. The spatial distribution of sensors may enable the device to create a detailed temperature map of the ear canal and tympanic membrane. By utilizing multiple sensing points, the device may be able to detect localized temperature variations that could be indicative of infection. This grid pattern arrangement may also help mitigate the effects of slight movements or positioning errors during examination, potentially improving the reliability and consistency of temperature readings across different areas of the ear canal.

The grid array infrared sensor's design may incorporate multiple infrared sensors arranged in an x-y grid pattern, which may help mitigate the need for precise positioning within the ear canal. This arrangement may allow the device to capture temperature readings from a wider area, potentially reducing the impact of slight misalignments during use. The x-y grid pattern may enable the device to create a two-dimensional thermal map of the ear canal, providing a more comprehensive view of temperature distribution. This feature may be particularly beneficial in clinical settings where quick and accurate readings are essential, as it may reduce the time and skill required to position the device correctly while maintaining measurement accuracy.

The device may include a display unit with a user interface designed to provide real-time infection detection readings. This feature may allow healthcare professionals to receive immediate feedback on potential ear infections as the examination is being conducted. The real-time display may show temperature readings, thermal maps, or other visual representations of the data collected by the grid array infrared sensor. This immediate access to diagnostic information may enable quicker decision-making and potentially more timely treatment initiation. The user interface may also include interactive elements that allow the examiner to adjust settings, zoom in on specific areas of interest, or toggle between different data visualization modes, enhancing the device's utility in clinical settings.

The acute otitis media ear infection detection device may also be designed as a handheld unit, which may offer several advantages in clinical use. Its portable nature may allow for easy maneuverability during ear examinations, potentially improving the ease and speed of diagnostic procedures. The handheld design may also facilitate use in various healthcare settings, from traditional clinics to mobile health units or home care scenarios. The compact form factor may contribute to the device's versatility, allowing healthcare providers to easily transport and use it in different examination rooms or during house calls. This handheld configuration may also enhance user control and precision during the examination process, potentially improving the accuracy of temperature readings and infection detection.

The grid array infrared sensor in the device may be designed to adapt to variations in ambient temperature, which may help maintain accuracy in infection detection across different environmental conditions. This adaptive capability may involve real-time calibration or compensation algorithms that adjust the sensor's readings based on the surrounding temperature. By accounting for ambient temperature fluctuations, the device may provide more consistent and reliable measurements, regardless of the examination environment. This feature may be particularly valuable in settings where temperature control is challenging, such as in emergency rooms, field clinics, or home care situations. The ability to adapt to ambient temperature variations may enhance the device's versatility and reliability across a wide range of clinical scenarios.

The grid array infrared sensor may also be integrated into a multi-functional healthcare device, which may be designed for use by both healthcare providers and patients. This integration may allow the device to serve multiple diagnostic or monitoring purposes beyond ear infection detection. For healthcare providers, the multi-functional nature may streamline examinations by combining several diagnostic tools into one device, potentially improving efficiency in clinical settings. For patients, the device may offer the capability for home monitoring of ear health, potentially enabling earlier detection of infections or tracking the progress of treatment. The versatility of this multi-functional design may enhance the device's value in various healthcare contexts, from professional clinical use to telemedicine applications and patient self-monitoring.

The device may also include additional operations that enhance its reliability and user awareness. It may perform self-diagnostic operations, which could involve routine checks of sensor functionality, calibration status, and overall system health. These self-diagnostics may help ensure the device maintains its accuracy and performance over time. The device may also be capable of detecting sensor malfunctions, which could include issues such as sensor drift, damage, or calibration errors. Upon detecting any malfunctions, the device may output alerts to notify the user. These alerts could be visual, auditory, or haptic, ensuring that the user is promptly informed of any issues that might affect the device's performance or the accuracy of its readings. This combination of self-diagnostics, malfunction detection, and alert systems may contribute to the device's reliability and may help maintain the integrity of ear infection diagnoses.

The grid array infrared sensor in the device may be tuned to minimize energy consumption, which may help extend the battery life of the acute otitis media ear infection detection device. This energy-efficient design may involve optimizing the sensor's power draw, implementing sleep modes when not in active use, or using adaptive sampling rates based on the examination needs. By reducing energy consumption, the device may be able to operate for longer periods between charges or battery replacements, potentially increasing its utility in clinical settings or for extended use scenarios. The extended battery life may also enhance the device's portability and reliability, especially in situations where frequent recharging might be inconvenient or impossible. This energy-efficient tuning may contribute to the overall usability and practicality of the device in various healthcare environments.

The device may also incorporate additional operations that enhance its diagnostic capabilities. It may perform thermal mapping of the ear canal, creating a detailed temperature profile of the entire examined area. This thermal map may provide a comprehensive view of temperature distribution, potentially highlighting areas of inflammation or infection. The device may also measure the overall temperature of the ear canal, providing a baseline for comparison. Additionally, the device may include compensation mechanisms for earwax or other occlusions that could interfere with accurate readings. This compensation may involve algorithms that adjust temperature calculations based on detected obstructions or alternative measurement techniques that can bypass minor blockages. These combined operations may improve the device's ability to provide accurate diagnoses even in less-than-ideal examination conditions, potentially enhancing its reliability and effectiveness in detecting ear infections.

The device may also include a secondary sensor specifically designed to detect physical obstructions in the ear canal. This additional sensor may use various technologies such as ultrasound, optical sensing, or mechanical pressure detection to identify the presence of earwax, foreign objects, or structural abnormalities that could interfere with accurate temperature readings. By detecting these obstructions, the device may be able to alert the user to potential examination difficulties or inaccuracies. This feature may help ensure that the primary grid array infrared sensor has a clear path for temperature measurement, potentially improving the reliability of infection detection. The secondary sensor may also provide valuable additional diagnostic information, helping healthcare providers assess the overall condition of the ear canal beyond just temperature-related indicators of infection.

The device may also perform additional operations that enhance its diagnostic capabilities. It may identify specific ear canal temperatures and infrared data from the collected sensor data, providing detailed thermal information about different areas within the ear. Using this information, the device may generate a comprehensive thermal map of the ear canal. This map may visually represent temperature variations across the examined area, potentially using color coding or other graphical elements to highlight areas of concern. These areas of concern may include regions with elevated temperatures, unusual temperature gradients, or other thermal patterns associated with infection or inflammation. By generating this detailed thermal map, the device may provide healthcare professionals with a visual tool for quickly identifying potential infection sites and assessing the extent of any detected issues, potentially aiding in more accurate and efficient diagnoses.

The handheld infrared temperature sensing device for diagnosing acute otitis media may incorporate several advanced features. It utilizes one or more processors to manage its operations and includes a sensor array capable of measuring tympanic temperatures across multiple points on the tympanic membrane. This multi-point measurement capability may allow for a more comprehensive assessment of ear temperature compared to single-point thermometers. The device also features a display unit, which may provide visual feedback of the diagnostic results. The non-transitory computer-readable media stores instructions that enable key functionalities when executed by the processors. These include receiving sensor data from the array, mapping the temperature distribution across the tympanic membrane, detecting signs of acute otitis media based on this thermal mapping, and displaying the detection results directly to the patient. This combination of features may allow for rapid, patient-friendly diagnostics, potentially improving the speed and comfort of ear infection detection.

The sensor array in this device may be designed to measure temperatures from multiple regions of the ear simultaneously. This capability may allow for a more comprehensive thermal profile of the ear, potentially capturing temperature variations that might be missed by single-point measurements. Additionally, the device correlates these tympanic temperature readings with axillary temperatures. This correlation may provide a more robust diagnostic approach, as it compares the localized ear temperatures with a general body temperature indicator. By establishing this relationship between tympanic and axillary temperatures, the device may be able to more accurately identify abnormal temperature elevations specific to ear infections, potentially improving the accuracy of acute otitis media diagnoses. This multi-region measurement and temperature correlation approach may enhance the device's ability to differentiate between systemic fevers and localized ear infections.

The sensor array in this device may also incorporate multiple individual sensors arranged in a spatially distributed grid pattern. This configuration may allow for comprehensive coverage of the tympanic membrane and surrounding areas, capturing thermal data from various points simultaneously. The grid pattern arrangement may enable the creation of detailed temperature maps, potentially revealing subtle temperature variations across the examined area. This distributed sensing approach may help mitigate issues related to sensor positioning, as it captures data from a wider area rather than relying on a single point of measurement. The spatial distribution of sensors may also contribute to the device's ability to detect localized hot spots or unusual temperature gradients that could be indicative of infection, potentially enhancing the accuracy and reliability of acute otitis media diagnoses.

The sensor array in this device may also be designed to adapt to variations in ambient temperature, which may help maintain accuracy in infection detection across different environmental conditions. This adaptive capability may involve real-time calibration or compensation algorithms that adjust the sensor readings based on the surrounding temperature. By accounting for ambient temperature fluctuations, the device may provide more consistent and reliable measurements, regardless of the examination environment. This feature may be particularly valuable in settings where temperature control is challenging, such as in emergency rooms, field clinics, or home care situations. The ability to adapt to ambient temperature variations may enhance the device's versatility and reliability across a wide range of clinical scenarios, potentially ensuring accurate acute otitis media diagnoses in various settings.

The sensor array may be integrated into a multi-functional healthcare device designed for patient use. This integration may allow the device to serve multiple diagnostic or monitoring purposes beyond ear infection detection. For patients, this may offer the capability for home monitoring of ear health, potentially enabling earlier detection of infections or tracking the progress of treatment. The multi-functional nature of the device may include features such as general temperature monitoring, symptom tracking, or even telemedicine capabilities. This versatility may enhance the device's value for patients, potentially improving their ability to manage their health between clinical visits. The integration of the acute otitis media detection functionality into a broader healthcare device may also contribute to patient engagement and proactive health management, potentially leading to earlier interventions and improved outcomes in ear infection cases.

The device may incorporate additional operations that enhance its reliability and user awareness. It may perform self-diagnostic operations, which could involve routine checks of sensor functionality, calibration status, and overall system health. These self-diagnostics may help ensure the device maintains its accuracy and performance over time. The device may also be capable of detecting malfunctions, which could include issues such as sensor drift, damage, or calibration errors. Upon detecting any malfunctions, the device may output alerts to notify the user. These alerts could be visual, auditory, or haptic, ensuring that the user is promptly informed of any issues that might affect the device's performance or the accuracy of its readings. This combination of self-diagnostics, malfunction detection, and alert systems may contribute to the device's reliability and may help maintain the integrity of acute otitis media diagnoses, particularly important for a patient-use device.

The device may include a secondary sensor specifically designed to detect physical obstructions on the tympanic membrane. This additional sensor may use various technologies such as ultrasound, optical sensing, or mechanical pressure detection to identify the presence of earwax, foreign objects, or structural abnormalities that could interfere with accurate temperature readings. By detecting these obstructions, the device may be able to alert the user to potential examination difficulties or inaccuracies. This feature may help ensure that the primary sensor array has a clear view of the tympanic membrane for temperature measurement, potentially improving the reliability of infection detection. The secondary sensor may also provide valuable additional diagnostic information, helping patients assess the overall condition of their ear canal beyond just temperature-related indicators of infection, potentially guiding them on when to seek professional medical attention.

The device may perform additional operations that enhance its diagnostic capabilities. It may identify specific ear canal temperatures and infrared data from the collected sensor data, providing detailed thermal information about different areas within the ear. Using this information, the device may generate a comprehensive thermal map of the ear canal. This map may visually represent temperature variations across the examined area, potentially using color coding or other graphical elements to highlight areas of concern. These areas of concern may include regions with elevated temperatures, unusual temperature gradients, or other thermal patterns associated with infection or inflammation. By generating this detailed thermal map, the device may provide patients with a visual tool for quickly identifying potential infection sites and assessing the extent of any detected issues. This feature may be particularly valuable for patient use, as it could help individuals make more informed decisions about seeking medical care for potential ear infections.

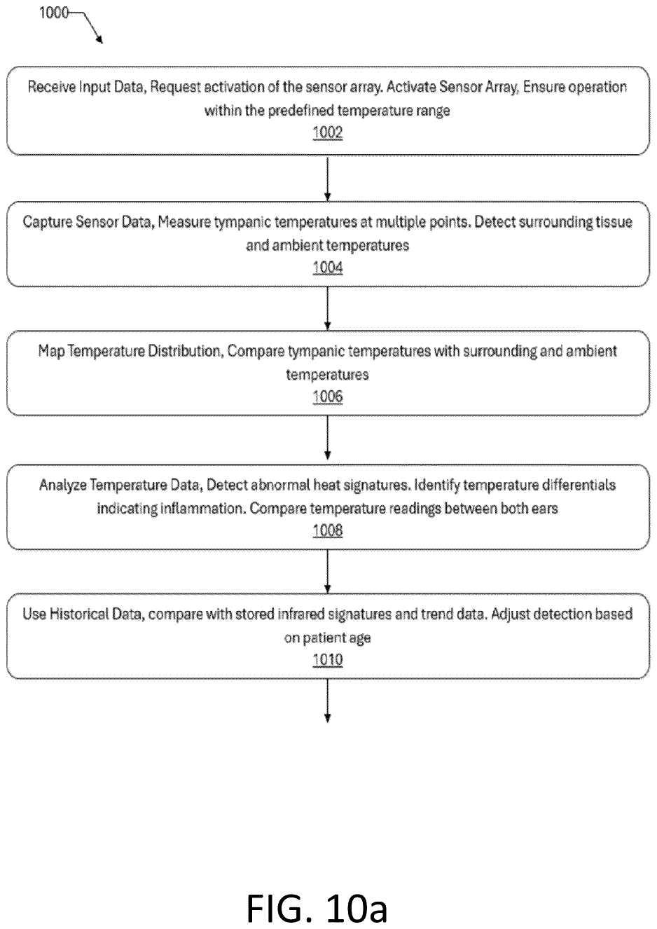

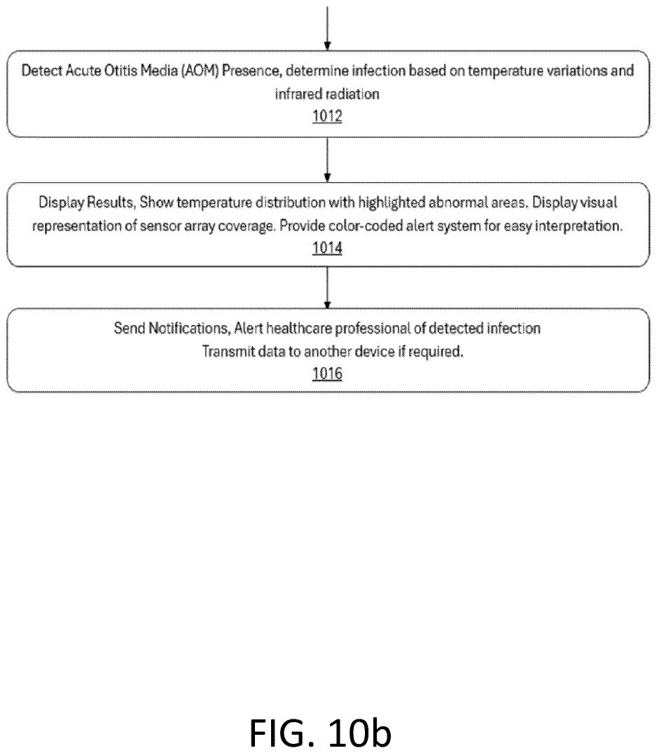

FIGS. 10a-b present an algorithmic flow diagram that outlines the data collection and operational workflow of the ear infection detection device. The order in which the operations or steps are described is not intended to be construed as a limitation, and any number of the described operations may be combined in any order and/or in parallel to implement process 1000.

At block 1002, the process 1000 may include initial stage of the data collection and operational workflow for the ear infection detection device. This block may encompass the activation and initialization of the device's sensing capabilities, particularly the Grid Sensor (101). The process at this stage may involve powering up the sensor array, performing initial calibrations, and preparing the system to receive incoming thermal data from the ear canal and tympanic membrane. The initialization process may include setting up communication protocols between the Grid Sensor (101) and the Microprocessor(s) (109), ensuring that the data pathway is clear and ready for transmission. This stage may also involve configuring the sensor's sensitivity parameters, which may be adjustable based on ambient conditions or user preferences. In some implementations, block 1002 may incorporate a self-diagnostic routine to verify the proper functioning of all sensor elements within the grid array. This could involve running a quick test sequence to confirm that each individual infrared detection element is operational and accurately calibrated. The process at this block may also include initializing the device's memory systems to prepare for data storage and retrieval operations that will occur during the subsequent steps of the workflow. This may involve clearing temporary storage buffers and loading any necessary reference data or calibration parameters from long-term memory. Additionally, Block 1002 may trigger the activation of user interface components, such as the LCD, Touch Screen, and LED indicators (113), to provide visual feedback that the device is powered on and ready for use. This could include displaying a start-up screen or running through a brief user interface check to ensure all display and input elements are functioning correctly.

At block 1004, the process 1000 may include initiating the data collection process for the ear infection detection device. This block may interface directly with the Grid Sensor (101) to gather infrared temperature readings from multiple points across the ear canal and tympanic membrane. The captured data may be in the form of raw voltage signals that correspond to detected infrared radiation. This block may also involve initial signal conditioning and analog-to-digital conversion to prepare the data for further processing. The captured sensor data is then passed to subsequent blocks in the workflow for analysis and interpretation.

At block 1006, the process 1000 may include processing the captured sensor data to create a structured thermal profile of the scanned area. This block may utilize the spatial information provided by the Grid X Axis (120) and Grid Y Axis (121) to associate temperature readings with specific locations within the ear canal. The mapping process may involve interpolation techniques to generate a continuous temperature distribution from discrete sensor points. The resulting temperature map may be stored in the Memory (128) for further analysis and may serve as input for the graphical representation of thermal patterns in later stages of the workflow.

At block 1008, the process 1000 may include performing in-depth analysis of the mapped temperature distribution to detect variations indicative of irregularities. This block may employ statistical methods and pattern recognition algorithms to identify temperature anomalies. It may compare the current readings against predefined thresholds or typical temperature ranges for healthy ear canals. The analysis process may leverage the computational capabilities of the Microprocessor(s) (109) to perform complex calculations. The results of this analysis may be used to flag potential areas of concern for further evaluation in subsequent blocks.

At block 1010, the process 1000 may include incorporating temporal context. This block may access previously stored temperature profiles from the Memory (128) to perform trend analysis. It may compare current readings with historical data to identify significant changes over time. This block may also include adaptive algorithms that adjust detection parameters based on observed patterns, potentially improving the accuracy of infection detection over multiple uses. The comparative analysis may help in distinguishing between normal variations and clinically significant temperature changes.

At block 1012, the process 1000 may include identifying specific markers associated with ear infections. This block may utilize the results from previous analysis blocks to make a determination about the presence of acute otitis media. It may employ decision-making algorithms that consider multiple factors such as temperature differentials, spatial patterns, and historical comparisons. The detection process may involve machine learning models trained on clinical datasets to recognize infection-related thermal signatures. This block may generate a confidence score or probability estimate for the presence of an infection.

At block 1014, the process 1000 may include presenting the diagnostic findings to the user through the device's interface. This block may interact with the LCD, Touch Screen, and LED indicators (113) to provide visual feedback. It may generate graphical representations such as color-coded heat maps or simplified diagnostic indicators. The display process may involve data formatting and graphical rendering to ensure clear and intuitive presentation of complex thermal data. This block may also trigger auditory or haptic alerts through the User Interface (111) components to draw attention to significant findings.

At block 1016, the process 1000 may include facilitating the sharing of diagnostic information beyond the device itself. This block may utilize the Data Transfer, Wi-Fi, Bluetooth, and Cellular modules (127) to securely transmit processed data and results. It may interface with electronic health record systems or telemedicine platforms to integrate the diagnostic findings into broader patient care contexts. The transmission process may involve data encryption and compression to ensure secure and efficient data transfer. This block may also receive external data or software updates, potentially enhancing the device's diagnostic capabilities over time.

The process 1000 may also include any or all of the following operations and/or details.

The infrared temperature sensing device for diagnosing acute otitis media may be designed for use by healthcare professionals. It incorporates a sensor array that measures tympanic temperatures at multiple points on a patient's tympanic membrane. The device processes this data to map the temperature distribution across the membrane, which is then analyzed to detect signs of acute otitis media. A key feature is the display unit that presents this temperature distribution to the healthcare professional, with areas of abnormal heat highlighted to indicate potential ear infections. This visual representation aids in quick and accurate diagnosis.

The device may enhance diagnostic clarity by providing a visual representation of the sensor array's coverage within the patient's ear canal. This feature allows healthcare professionals to ensure proper positioning and comprehensive data collection during the examination, potentially improving the accuracy of the diagnosis.

The device may include a user-activated sensor array feature. Upon receiving input data requesting activation, the device initiates the sensor array, allowing healthcare professionals to control when temperature measurements are taken. This feature may help conserve power and ensure measurements are taken at the most appropriate times during an examination.

The sensor array in the device may be designed to operate within a predetermined temperature range. This specification ensures that the device can accurately capture and interpret temperature data within the expected range for diagnosing ear infections, potentially improving the reliability of the measurements and subsequent diagnoses.

The device's diagnostic capabilities may be enhanced by its ability to detect abnormal heat signatures caused by tympanic membrane inflammation. This feature allows for more precise identification of potential infections, as inflammation is a key indicator of acute otitis media. The detection of these abnormal heat patterns plays a crucial role in the overall diagnostic process.

The device's diagnostic approach may be further refined by its ability to detect surrounding temperatures in the ear canal. By generating temperature differentials between the tympanic membrane and surrounding areas, the device can provide a more comprehensive thermal profile. These temperature differentials are then utilized in the detection of acute otitis media, potentially improving diagnostic accuracy.

The device may incorporate ambient temperature detection near the tympanic membrane as part of its diagnostic process. By generating temperature differentials between the ambient and tympanic membrane temperatures, the device can account for environmental factors that might influence readings. These differentials are then used in the detection of acute otitis media, potentially enhancing the accuracy of the diagnosis.

The method for detecting acute otitis media ear infection described herein may involve receiving sensor data from a device's sensor array and mapping the temperature distribution of the tympanic membrane. This temperature distribution is then analyzed to detect signs of infection. The method includes displaying this distribution to a healthcare professional, with areas of abnormal heat highlighted, facilitating a visual diagnosis of potential ear infections.

The method may incorporate the detection of infrared radiation corresponding to elevated temperatures in the ear canal. This feature allows for the identification of potential infections based on thermal indicators, enhancing the overall diagnostic capabilities of the device and method.

The method may also include tuning the sensor array to specifically detect infrared radiation emitted from an inflamed tympanic membrane, distinguishing it from surrounding ear canal tissues. This targeted approach may improve the accuracy of infection detection by focusing on the most relevant thermal indicators.

The method may incorporate historical data analysis by storing trend data of heat patterns associated with past infections. This stored information is then used in conjunction with current sensor data to detect new infections, potentially improving diagnostic accuracy by leveraging patterns observed in previous cases.

The method may utilize a database of specific infrared signatures associated with ear canal infections. These stored signatures are compared with current sensor data to detect infections, potentially allowing for more precise identification of different types or stages of ear infections based on their unique thermal profiles.

The method may enhance its diagnostic capabilities by identifying and detecting both heat and infrared radiation variations indicative of infection. By analyzing these specific variations in the sensor data, the method can more accurately identify the presence of an ear infection, potentially improving the overall reliability of the diagnosis.

The method may include a calibration process for the sensor array, specifically designed to detect ear infections by identifying a predefined increase in ear canal temperature. This calibration ensures that the device can accurately recognize the thermal thresholds associated with infections, potentially improving diagnostic consistency across different examinations and patients.

The method may incorporate a notification system that sends alert data to another device operated by the healthcare professional. This feature ensures that the professional is promptly informed when an infection is detected, even if they are not directly interacting with the primary diagnostic device at the time of detection.

In examples, the device described herein may be a healthcare professional's device that may be equipped with a sensor array capable of measuring tympanic temperatures across multiple points on a patient's tympanic membrane. It processes this sensor data to map the temperature distribution of the tympanic membrane and uses this information to detect signs of acute otitis media. This comprehensive approach to temperature mapping and analysis may provide healthcare professionals with detailed insights for accurate diagnoses.

The healthcare professional's device may include a display feature that visually presents the mapped temperature distribution of the tympanic membrane. This display highlights areas of abnormal heat that may indicate ear infections, providing an intuitive visual aid for healthcare professionals to quickly identify potential issues during examinations.

The device may incorporate a comparative analysis feature that examines temperature readings from both ears of a patient. By detecting temperature differences of at least 0.5° C. between the ears, the device provides an additional indicator for identifying acute otitis media. This bilateral comparison may enhance the accuracy of diagnoses by highlighting asymmetrical thermal patterns.

The device may utilize age-specific trend data to enhance its diagnostic capabilities. By storing infection trend data for various age ranges and allowing input of the patient's age, the device can apply the most relevant historical data to the current examination. This age-tailored approach may improve the accuracy of acute otitis media detection across different patient demographics.

The healthcare professional's device may feature a color-coded alert system displayed to the user, indicating the presence or absence of acute otitis media. This intuitive visual feedback system may allow for quick and clear communication of diagnostic results, potentially streamlining the examination process and facilitating prompt treatment decisions.

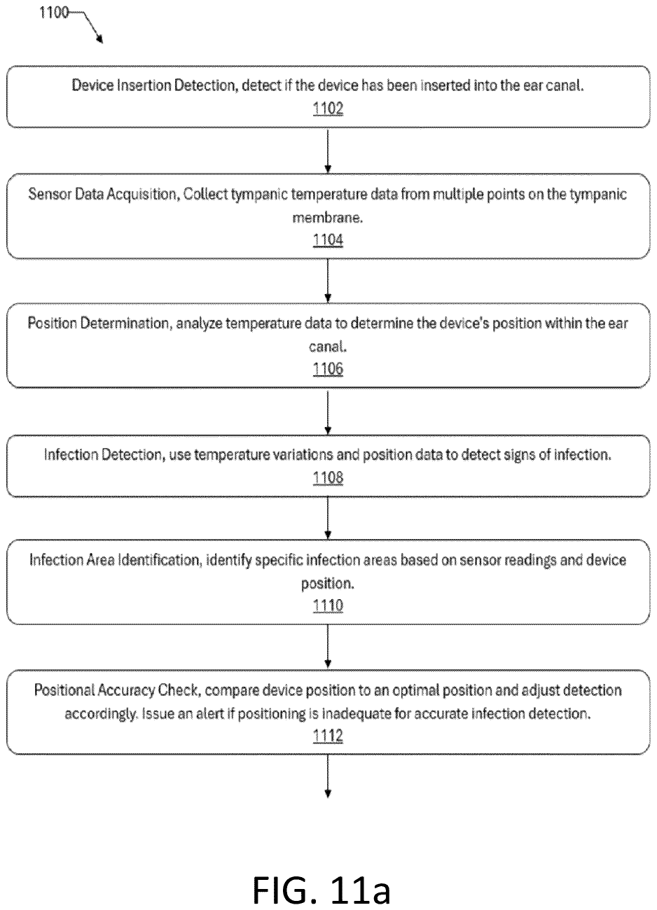

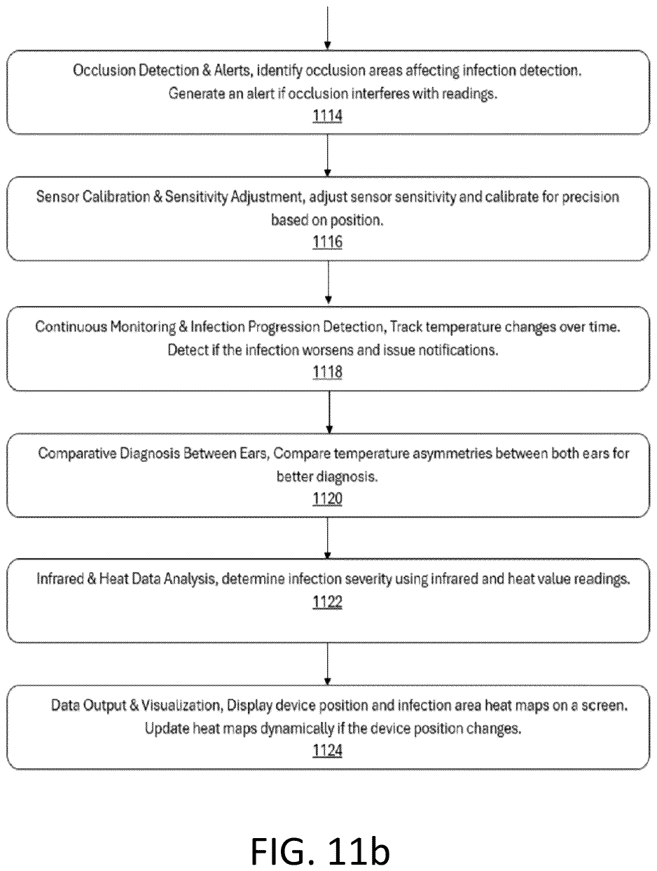

FIGS. 11a-b provides an algorithmic flow diagram (1100) illustrating the sequence of operations involved in inserting the device into the ear canal. The order in which the operations or steps are described is not intended to be construed as a limitation, and any number of the described operations may be combined in any order and/or in parallel to implement process 1100.

At block 1102, the process 1100 may include initiating and managing the collection of thermal data from the Grid Sensor (101). This process may also include detecting if the device has been inserted into the ear canal.

At block 1104, the process 1100 may include gathering infrared temperature readings from multiple points across the ear canal and tympanic membrane. The captured data may be in the form of raw voltage signals that correspond to detected infrared radiation. This block may also involve initial signal conditioning and analog-to-digital conversion to prepare the data for further processing. The Sensor Data Acquisition block serves as the primary input stage for the entire diagnostic process, providing the raw thermal information that subsequent blocks will analyze and interpret.

At block 1106, the process 1100 may include ascertaining the precise location of the device within the ear canal. This block may utilize spatial data from the Grid X Axis (120) and Grid Y Axis (121) in conjunction with the temperature readings to map the device's position relative to the ear anatomy. The position information is crucial for ensuring accurate temperature mapping and may be used to adjust subsequent analysis parameters. This block may interact with the Sensor Data Acquisition block to correlate temperature readings with specific anatomical locations, and may provide feedback to the user interface to guide proper device placement.

At block 1108, the process 1100 may include identifying potential ear infections. This block may employ advanced algorithms to analyze the temperature distribution data, looking for patterns and anomalies indicative of infection. It may compare the processed thermal data against predefined thresholds or utilize machine learning models trained on clinical datasets to recognize infection-related thermal signatures. The Infection Detection block may interface with the Memory (128) to access historical data or reference infection patterns, and may generate a confidence score or probability estimate for the presence of an infection.

At block 1110, the process 1100 may include pinpointing precise locations within the ear canal that show signs of infection. This block may utilize the spatial mapping data from the Position Determination block in conjunction with the analysis results from the Infection Detection block to create a detailed map of potentially infected areas. It may employ image processing techniques to highlight and isolate regions of interest within the thermal profile. The output from this block may be used to generate targeted visual representations for the user interface and may inform more focused analysis in subsequent diagnostic steps.

At block 1112, the process 1100 may include ensuring the reliability of the diagnostic process by verifying the correct positioning of the device. This block may compare the determined position from the Position Determination block against optimal positioning parameters stored in memory. If the device's position deviates from the ideal range, this block may trigger alerts through the User Interface (111) components, potentially including visual, auditory, or haptic feedback to guide the user in adjusting the device's placement. This block plays a crucial role in maintaining the accuracy and consistency of the diagnostic measurements.

At block 1114, the process 1100 may include identifying and responding to potential obstructions in the ear canal that may interfere with accurate temperature readings. This block may analyze the thermal profile and signal strength data to detect anomalies that suggest the presence of earwax, foreign objects, or other occlusions. Upon detecting an occlusion, this block may trigger alerts through the User Interface (111) to notify the user of the potential interference. It may also adjust the analysis parameters in other blocks to compensate for the detected occlusion, ensuring the most accurate diagnosis possible under the given conditions.

At block 1116, the process 1100 may include maintaining the accuracy and reliability of the sensor array throughout the diagnostic process. This block may continuously monitor the incoming data from the Sensor Data Acquisition block and environmental factors to dynamically adjust the sensitivity and calibration of the Grid Sensor (101). It may employ adaptive algorithms that fine-tune the sensor parameters based on real-time data, ensuring optimal performance across varying conditions. This block may interface with the Memory (128) to store and retrieve calibration data, and may communicate with other blocks to ensure that any adjustments are factored into their respective analyses.

At block 1118, the process 1100 may include enabling ongoing assessment of the ear canal's thermal conditions throughout the examination. This block may repeatedly cycle through the data acquisition and analysis processes, providing real-time updates on temperature variations and potential infection indicators. It may interface with the Memory (128) to track changes over time and may trigger alerts through the User Interface (111) if significant changes are detected. The Continuous Monitoring block enhances the device's ability to capture dynamic thermal patterns and may improve the accuracy of infection detection by observing temperature trends rather than single-point measurements.

At block 1120, the process 1100 may include enhancing the diagnostic accuracy by contextualizing current readings against historical or reference data. This block may access previously stored temperature profiles from the Memory (128) to perform trend analysis, comparing current readings with historical data to identify significant changes over time. It may also compare the current thermal profile against a database of known infection patterns to improve diagnostic confidence. The Comparative Diagnosis block may interface with the Infection Detection and Specific Area Identification blocks to refine their outputs based on comparative analysis results.

At block 1122, the process 1100 may include processing the raw infrared data collected by the Grid Sensor (101) to extract meaningful thermal information. This block may employ specialized algorithms to interpret the infrared signals, converting them into accurate temperature readings and thermal distribution maps. It may apply noise reduction techniques, compensate for ambient temperature variations, and perform spatial interpolation to generate high-resolution thermal profiles. The Infrared Data Analysis block provides critical input to other analytical blocks in the system and may interface with the display components to generate detailed thermal visualizations.

At block 1124, the process 1100 may include presenting the diagnostic findings in a clear and intuitive format. This block may generate various visual representations of the thermal data, such as color-coded heat maps, 3D temperature models, or simplified diagnostic indicators. It interfaces directly with the LCD, Touch Screen, and LED indicators (113) to render these visualizations. The block may also format numerical data and diagnostic conclusions for display, ensuring that complex thermal information is presented in an easily interpretable manner for healthcare professionals. Additionally, this block may prepare data for external transmission via the Data Transfer modules (127), facilitating integration with broader healthcare information systems.

The process 1100 may also include any or all of the following operations and/or details.

The innovative device described herein for detecting ear infections may incorporate advanced sensor technology and processing capabilities. This apparatus features a sensor array designed to measure tympanic temperatures at multiple points on a patient's tympanic membrane. Powered by one or more processors and guided by instructions stored in non-transitory computer-readable media, the device performs a series of operations upon insertion into a patient's ear canal. It receives sensor data containing temperature information, determines its position within the ear canal, and utilizes this data to detect signs of infection. By considering both the sensor readings and the device's position, it can accurately identify specific areas of infection within the ear canal, providing a comprehensive analysis of the patient's condition.

The ear infection detection device may incorporate an advanced positioning system that enhances its diagnostic accuracy. This system determines the degree of variance between the actual position of the device within the ear canal and its optimal position. By utilizing this positional information, the device refines its ability to identify infection areas, ensuring that the diagnostic results are adjusted based on the precise location of the sensor array relative to the tympanic membrane and surrounding tissues.

To improve diagnostic reliability, the ear infection detection device may include sophisticated occlusion detection capabilities. The system can identify areas of obstruction within the ear canal, such as excessive earwax or foreign objects. It then assesses how these occlusions may impact the accuracy of infection detection. If the device determines that an occlusion significantly affects its ability to detect infections, it promptly alerts the user, ensuring that potential limitations in the diagnostic process are clearly communicated.

The ear infection detection device may feature dynamic sensor calibration to maintain optimal performance. The system can adjust the sensitivity of its sensor array in real-time, fine-tuning its detection capabilities based on the specific conditions encountered during each examination. This adaptive calibration process ensures that the device maintains precise detection accuracy across various patient scenarios and environmental conditions, enhancing the reliability of its diagnostic results.

Continuous monitoring capabilities may be integrated into the ear infection detection device to track the progression of infections over time. The system monitors temperature readings throughout the examination period, analyzing trends and changes in thermal patterns. If the device detects signs that an infection has worsened, such as increasing temperature or expanding areas of abnormal heat, it promptly notifies the user. This feature enables healthcare professionals to make informed decisions about treatment adjustments or follow-up care based on real-time data.

The ear infection detection device may also employ a comparative analysis approach to enhance diagnostic accuracy. By examining and comparing temperature readings from both ears of a patient, the system can detect thermal asymmetries that may indicate the presence of an infection. This bilateral comparison allows the device to identify subtle differences in temperature patterns between the ears, providing an additional layer of diagnostic information that may help differentiate between localized infections and normal variations in body temperature.

Advanced data analysis capabilities may be incorporated into the ear infection detection device to provide a more comprehensive assessment of infection severity. The system extracts both heat and infrared values from the sensor data, utilizing these distinct but related measurements to evaluate the intensity of the infection. By analyzing the relationship between these values, the device can offer insights into the severity of the detected infection, potentially aiding healthcare professionals in determining appropriate treatment strategies.

A specialized device as described herein may be utilized to minimize positional inaccuracy in ear infection detection, incorporating advanced sensor technology and sophisticated processing capabilities. This apparatus utilizes a sensor array to measure tympanic temperatures across multiple points on a patient's tympanic membrane. Guided by one or more processors and instructions stored in non-transitory computer-readable media, the device performs a series of operations upon insertion into the ear canal. It receives and processes sensor data, determines its position within the ear canal, and uses this information to accurately detect signs of infection and identify specific infection areas, all while accounting for its spatial orientation.

The device for minimizing positional inaccuracy in ear infection detection may utilize a grid-based infrared temperature sensor. This advanced sensor configuration allows for precise spatial mapping of temperature variations across the tympanic membrane and ear canal. The grid-based design enables the device to capture a comprehensive thermal profile of the examined area, potentially improving the accuracy and resolution of infection detection by providing detailed temperature data from multiple points simultaneously.

To ensure optimal diagnostic accuracy, the ear infection detection device may include an alert system that notifies users when the device's position does not meet the required threshold for accurate measurements. This feature helps maintain the integrity of the diagnostic process by prompting users to adjust the device's placement if it falls outside the ideal range for reliable temperature readings. By providing immediate feedback on positioning, the system helps minimize errors that could result from suboptimal device orientation within the ear canal.

The ear infection detection device may employ an adaptive data selection process based on its position within the ear canal. The system determines which subset of temperature data is most relevant and reliable given its current orientation. This selective approach to data utilization allows the device to focus on the most accurate and pertinent information for identifying infection areas, potentially improving diagnostic precision by filtering out data that may be less reliable due to positioning factors.

A display unit may be integrated into the ear infection detection device to provide visual feedback on its position within the ear canal. This feature allows users to see a real-time representation of how the device is oriented during the examination. The visual guide may help healthcare professionals ensure proper placement and maintain optimal positioning throughout the diagnostic process, potentially improving the consistency and reliability of measurements across different examinations and patients.

The ear infection detection device may also include a display unit that visually represents identified infection areas within the ear canal. This feature translates complex thermal data into an intuitive visual format, allowing healthcare professionals to quickly locate and assess potential sites of infection. The visual representation may use color coding, heat maps, or other graphical elements to clearly delineate areas of concern, facilitating more informed and efficient diagnostic decisions.

An advanced display system may be incorporated into the ear infection detection device, capable of generating dynamic heat maps based on the device's position within the ear canal. Initially, the system produces a heat map reflecting the thermal profile detected from its starting position. If the device's position changes during the examination, the system updates the heat map in real-time, adjusting the visual representation to accurately reflect the new thermal data based on the altered orientation. This dynamic mapping capability ensures that healthcare professionals always have access to the most current and position-appropriate thermal information throughout the diagnostic process.

A method as described herein for detecting ear infections may utilize advanced sensor technology and data processing techniques. This approach involves inserting a specialized device into a patient's ear canal, which then collects temperature information via a sensor array capable of measuring tympanic temperatures across multiple points. The method determines the device's position within the ear canal and uses this positional data in conjunction with the temperature readings to detect signs of infection. By analyzing both the thermal profile and the spatial context, this method can accurately identify specific areas of infection within the ear canal, providing a comprehensive diagnostic assessment.

The method for detecting ear infections may incorporate a positioning accuracy assessment to enhance its diagnostic precision. This approach involves determining the degree of variance between the actual position of the detection device within the ear canal and its optimal position. By quantifying this positional discrepancy, the method can adjust its infection area identification process, ensuring that the diagnostic results account for any deviations from the ideal device placement and maintaining accuracy across various examination scenarios.

To improve diagnostic reliability, the ear infection detection method may include a sophisticated occlusion assessment process. This approach involves identifying areas of obstruction within the ear canal, such as excessive earwax or foreign objects, and evaluating how these occlusions may impact the accuracy of infection detection. If the method determines that an occlusion significantly affects its ability to detect infections, it generates an alert to notify the user of potential limitations in the diagnostic process, ensuring transparent communication of any factors that may influence the examination results.

The method for detecting ear infections may incorporate dynamic sensitivity adjustment of the sensor array based on the device's position within the ear canal. This adaptive approach allows the system to optimize its detection capabilities in real-time, fine-tuning the sensitivity of the sensors to account for variations in placement and ensure accurate temperature readings regardless of the exact orientation of the device. By adjusting sensor sensitivity in response to positional data, this method maintains high precision in its thermal measurements across different examination conditions.

Continuous monitoring capabilities may be integrated into the ear infection detection method to track the progression of infections over time. This approach involves ongoing analysis of temperature readings throughout the examination period, identifying trends and changes in thermal patterns. If the method detects signs that an infection has worsened, such as increasing temperatures or expanding areas of abnormal heat, it generates a notification to alert the user. This feature enables healthcare professionals to make informed decisions about treatment adjustments or follow-up care based on real-time data and observed changes in the patient's condition.

The ear infection detection method may employ a comparative analysis approach to enhance diagnostic accuracy. By examining and comparing temperature readings from both ears of a patient, the method can detect thermal asymmetries that may indicate the presence of an infection. This bilateral comparison allows for the identification of subtle differences in temperature patterns between the ears, providing an additional layer of diagnostic information that may help differentiate between localized infections and normal variations in body temperature. This comparative technique contributes to a more comprehensive and reliable assessment of the patient's ear health.

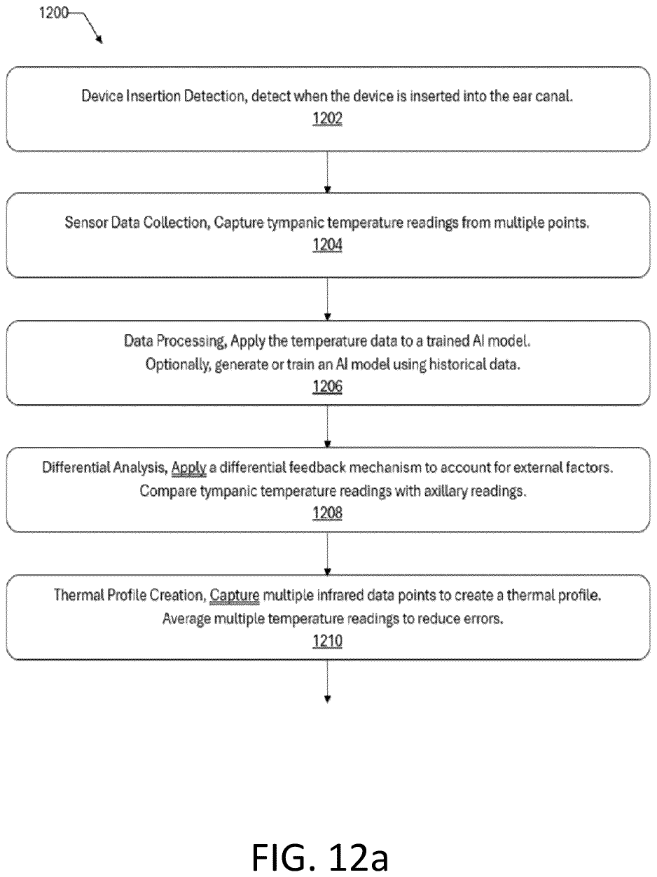

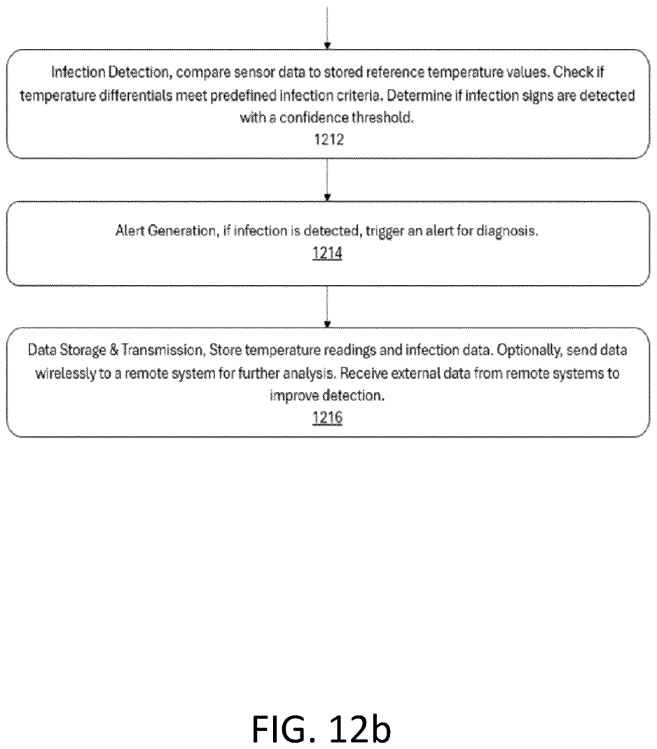

FIG. 12a-b presents an algorithmic flow diagram (1200) outlining the complete process of inserting the device into the ear, capturing sensor data, and executing advanced data processing techniques. The order in which the operations or steps are described is not intended to be construed as a limitation, and any number of the described operations may be combined in any order and/or in parallel to implement process 1200.

At block 1202, the process 1200 may include initiating the diagnostic process by identifying when the device is properly positioned within the ear canal. It may utilize data from the Grid Sensor to detect contact with the ear canal walls or changes in ambient temperature indicative of insertion. This block interfaces with the Data Collection and Temperature Readings block to trigger the start of data acquisition. The insertion detection mechanism may employ pressure sensors or infrared proximity detection to ensure accurate positioning, enhancing the overall reliability of subsequent temperature measurements.

At block 1204, the process 1200 may include managing the acquisition of thermal data from the Grid Sensor array. It coordinates the sampling of multiple infrared sensing points to create a comprehensive thermal profile of the ear canal and tympanic membrane. This block may implement adaptive sampling rates to balance measurement accuracy with power consumption. The collected data is then passed to the AI-Driven Machine Learning Algorithms block for advanced processing. The block may also perform initial data conditioning and error checking to ensure the quality of the raw sensor inputs.

At block 1206, the process 1200 may include applying sophisticated computational techniques to analyze the thermal data collected from the ear canal. It may utilize neural networks or other machine learning models trained on extensive clinical datasets to recognize patterns indicative of ear infections. This block interfaces with the Differential Analysis block to refine its outputs and may adapt its algorithms based on feedback from the Infection Detection with Confidence block. The AI-driven approach allows for continuous improvement in diagnostic accuracy as more data is processed over time.

At block 1208, the process 1200 may include performing comparative assessments of the temperature data to identify abnormal variations. It may calculate temperature gradients across different regions of the ear canal and compare these to established baselines for healthy and infected states. This block works in conjunction with the AI-Driven Machine Learning Algorithms to provide context-aware analysis. The results from this block feed into the Individualized Thermal Profiles Creation and Capture block, contributing to a more nuanced understanding of each patient's unique thermal characteristics.

At block 1210, the process 1200 may include generating and storing unique thermal signatures for each patient. It synthesizes data from the Differential Analysis and AI-Driven Machine Learning Algorithms to create comprehensive thermal maps of the ear canal. These profiles may be stored in the device's memory and used for future comparative analyses, enhancing the accuracy of repeat diagnoses. The block may also implement data compression techniques to efficiently manage the storage of these detailed thermal profiles.

At block 1212, the process 1200 may include making the final determination on the presence of an ear infection based on inputs from preceding analytical blocks. It may employ statistical methods to calculate a confidence score for the diagnosis, considering factors such as the consistency of thermal anomalies and their correlation with known infection patterns. This block interfaces with the Alert Generation block to trigger notifications based on the confidence level of the infection detection. It may also provide feedback to the AI-Driven Machine Learning Algorithms to refine future analyses.

At block 1214, the process 1200 may include notifying users of potential infections detected by the system. It translates the outputs from the Infection Detection with Confidence block into user-friendly alerts, which may include visual indicators on the device's display, audible alarms, or haptic feedback. This block may implement different alert levels based on the severity and confidence of the detected infection. It interfaces with the device's user interface components to ensure clear and timely communication of diagnostic results to healthcare professionals.

At block 1216, the process 1200 may include managing the post-processing handling of diagnostic data. It may utilize encryption protocols to securely transmit data to external systems such as electronic health records or telemedicine platforms. This block may also perform additional analysis on aggregated data to identify trends or patterns across multiple examinations. The secure storage component ensures that patient data is protected and compliant with relevant healthcare data regulations. This block may interface with cloud-based systems to enable remote access to diagnostic information by authorized healthcare providers.

The process 1200 may also include any and/or all of the following operations and/or details.

A computing device as described herein for detecting ear infections may incorporate one or more processors and a sensor array designed to measure tympanic temperatures across multiple points on a patient's tympanic membrane. The device's non-transitory computer-readable media contains instructions for several key operations. These include receiving an indication when the device is inserted into the ear canal, collecting temperature data from the sensor array, applying this data to a trained model specifically designed for infection detection, and ultimately detecting signs of infection based on the analysis of the data through the model.

This ear infection detection device may generate an artificial intelligence model specifically tailored for identifying infections within the ear canal. The sensor data collected by the device can then be applied directly to this AI model as part of the diagnostic process.

In some implementations, the device may train its artificial intelligence model using historical temperature data collected from other patients. This approach allows the device to leverage a broader dataset for more accurate diagnoses. The sensor data collected during an examination can then be applied to this pre-trained AI model, potentially improving the accuracy of infection detection.

The device may incorporate a differential calculation feedback mechanism designed to account for external factors that could affect the detection of acute otitis media infections. This mechanism can be applied to the sensor data collected during an examination, potentially helping to isolate infection-related temperature changes from other environmental or physiological factors.

Some versions of the device may include wireless communication capabilities. This feature allows the device to transmit subsequent sensor data collected over time to a remote system for further analysis. The device can then receive external data back from this remote system, which may be incorporated into the infection detection process, potentially providing additional context or comparative data to enhance diagnostic accuracy.

The ear infection detection device may utilize a machine learning model that has been specifically trained to recognize signs of infection from both infrared data and heat signature patterns. This specialized model can be applied to the sensor data collected during an examination, potentially allowing for more nuanced and accurate detection of ear infections.

In some implementations, the device may include a feature to determine if the detected signs of infection meet a predefined confidence threshold. If this threshold is met or exceeded, the device may output an alert specifically indicating a diagnosis of acute otitis media, providing clear and actionable information to healthcare providers.

A method for detecting ear infections may involve several key steps. It begins with receiving an indication that a device has been inserted into the ear canal. The method then collects temperature data from a sensor array within the device. This data is applied to a trained model designed specifically for detecting ear infections. Finally, the method uses the combination of the collected data and the model's analysis to detect signs of infection within the ear canal.

This ear infection detection method may involve simultaneous data capture from multiple infrared sensing points within the ear canal. By collecting data from various locations at once, the method can create a comprehensive thermal profile of the patient's tympanic membrane, providing a more detailed picture for analysis.

The method may include a comparative analysis of the sensitivity and specificity of tympanic temperature readings versus axillary temperature readings. This comparison can then be factored into the infection detection process, potentially allowing for more accurate diagnoses by considering multiple temperature measurement sites.

In some implementations, this comparative analysis method may achieve high levels of diagnostic accuracy. Specifically, it may yield a sensitivity of at least 91.7% and a specificity of at least 74.8% when diagnosing acute otitis media, representing a significant improvement in the reliability of ear infection detection.

The ear infection detection method may involve measuring temperatures in two distinct portions of the ear canal. By calculating the temperature differential between these areas and comparing it to preset criteria, the method can use this information as part of its infection detection process, potentially identifying localized areas of inflammation or infection. Doing so may include determining a first temperature reading of a first portion of the ear canal and determining a second temperature reading of a second portion of the ear canal. The process may then include determining a temperature differential of the first temperature reading and the second temperature reading. In these examples, detecting the signs of the infection is based at least in part on the temperature differential satisfying a pre-set temperature differential criteria.

Some versions of this method may incorporate a database of reference temperature values associated with ear infections. During the diagnostic process, the method can compare the collected sensor data against these stored reference values, using the similarities or differences to help determine the presence of an infection.

The method may enhance its accuracy by taking multiple temperature readings across the surface of the tympanic membrane. These readings can be averaged to reduce the impact of any individual measurement errors. This averaged value may then be used in the infection detection process, potentially providing a more reliable temperature assessment.

A system as described herein for detecting ear infections may incorporate processors and a specialized sensor array designed for measuring tympanic temperatures. The system includes instructions for several key functions: detecting when the device has been inserted into the ear canal, collecting temperature data, applying this data to a trained model, and ultimately detecting signs of infection based on the analysis.

This ear infection detection system may be capable of capturing data simultaneously from multiple infrared sensing points within the ear canal. This feature allows the system to create a detailed thermal profile of the patient's tympanic membrane, providing a comprehensive view for analysis.

The system may include functionality to compare the sensitivity and specificity of tympanic temperature readings against axillary temperature readings. This comparative analysis can be integrated into the infection detection process, potentially enhancing the accuracy of diagnoses by considering multiple temperature measurement sites.

In some implementations, this system's comparative analysis approach may achieve high levels of diagnostic accuracy. Specifically, it may yield a sensitivity of at least 91.7% and a specificity of at least 74.8% when diagnosing acute otitis media, representing a significant improvement in the reliability of ear infection detection.

The ear infection detection system may be designed to measure temperatures in two distinct portions of the ear canal. By calculating the temperature differential between these areas and comparing it to preset criteria, the system can use this information as part of its infection detection process, potentially identifying localized areas of inflammation or infection.

Some versions of this system may incorporate a database of reference temperature values associated with ear infections. During the diagnostic process, the system can compare the collected sensor data against these stored reference values, using the similarities or differences to help determine the presence of an infection.

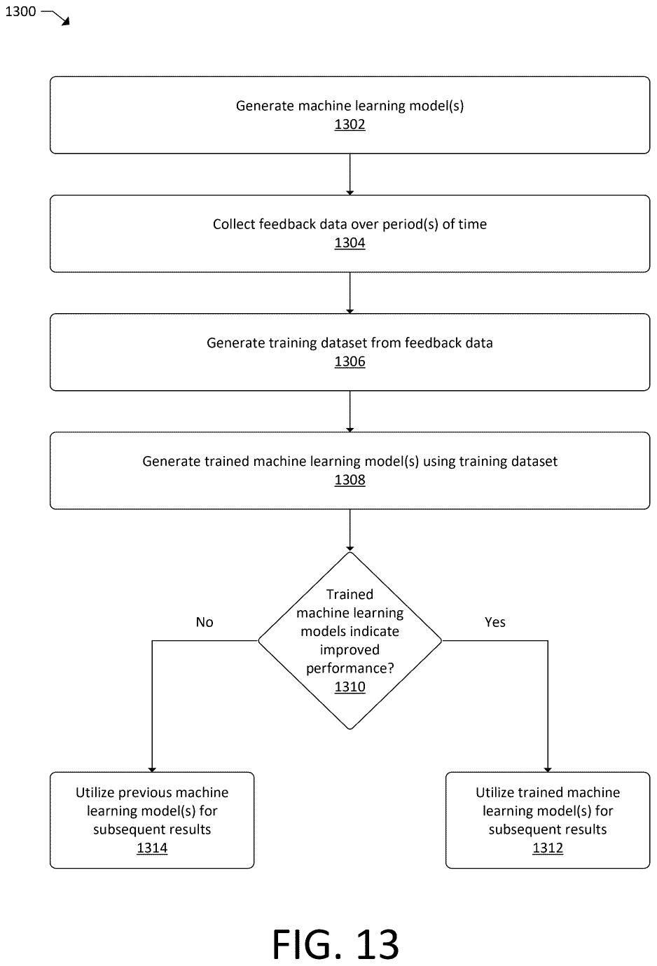

FIG. 13 is a flow diagram of an example process 1300 for the generation and training of artificial intelligence models (also referred to herein as machine learning models) to perform one or more of the processes described herein, according to an example described herein. The order in which the operations or steps are described is not intended to be construed as a limitation, and any number of the described operations may be combined in any order and/or in parallel to implement process 1300.

At block 1302, the process 1300 may include generating one or more artificial intelligence models, such as a machine learning model. A number of artificial intelligence techniques may be employed to generate and/or modify the layers and/or models described herein. Those techniques may include, for example, decision tree learning, association rule learning, artificial neural networks (including, in examples, deep learning), inductive logic programming, support vector machines, clustering, Bayesian networks, reinforcement learning, representation learning, similarity and metric learning, sparse dictionary learning, and/or rules-based artificial intelligence.

At block 1304, the process 1300 may include collecting feedback data over a period of time. The feedback data may include any data associated with determining questions and/or activities to present to a user, any described with respect to FIGS. 1-12b, or any other data that may be utilized to perform the operations described herein. This information may include, for example, user input data, user activity data, etc.

At block 1306, the process 1300 may include generating a training dataset from the feedback data. Generation of the training dataset may include formatting the feedback data into input vectors for the artificial intelligence model to intake, as well as associating the various data with the outcomes of the questions and/or activities described herein.

At block 1308, the process 1300 may include generating one or more trained artificial intelligence models utilizing the training dataset. Generation of the trained artificial intelligence models may include updating parameters and/or weightings and/or thresholds utilized by the models to determine appropriate questions to present to the user, appropriate activities to recommend, and the like.

At block 1310, the process 1300 may include determining whether the trained artificial intelligence models indicate improved performance metrics. For example, a testing group may be generated where the outcomes of given questions and/or activities are known but not to the trained artificial intelligence models. The trained artificial intelligence models may generate results, which may be compared to the known results to determine whether the results of the trained artificial intelligence model produce a superior result than the results of the artificial intelligence model prior to training.

In examples where the trained artificial intelligence models indicate improved performance metrics, the process 1300 may include, at block 1312, utilizing the trained artificial intelligence models for generating subsequent results. For example, the trained artificial intelligence models may be utilized to determine appropriate questions to present to the user, appropriate activities to recommend, appropriate account balances to be maintained, and/or the like. The trained artificial intelligence models may be utilized in any scenario where models are utilized as described herein.

In examples where the trained artificial intelligence models do not indicate improved performance metrics, the process 1300 may include, at block 1314, utilizing the previous iteration of the artificial intelligence models for generating subsequent results.

The present disclosure provides an overall understanding of the principles of the structure, function, manufacture, and use of the systems and methods disclosed herein. One or more examples of the present disclosure are illustrated in the accompanying drawings. Those of ordinary skill in the art will understand that the systems and methods specifically described herein and illustrated in the accompanying drawings are non-limiting embodiments. The features illustrated or described in connection with one embodiment may be combined with the features of other embodiments, including as between systems and methods. Such modifications and variations are intended to be included within the scope of the appended claims.

While the foregoing invention is described with respect to the specific examples, it is to be understood that the scope of the invention is not limited to these specific examples. Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Although the application describes embodiments having specific structural features and/or methodological acts, it is to be understood that the claims are not necessarily limited to the specific features or acts described. Rather, the specific features and acts are merely illustrative some embodiments that fall within the scope of the claims.

1. An infrared temperature sensing device for diagnosing acute otitis media by a healthcare professional, the infrared temperature sensing device comprising:
one or more processors;
a sensor array configured to measure tympanic temperatures across multiple points on a tympanic membrane of a patient;
a display unit;
a housing configured to be held by the healthcare professional and to contain the one or more processors and the sensor array, the housing having a distal end configured for at least partial insertion into an ear canal of the patient; and
non-transitory computer-readable media, housed in the housing, storing instructions that, when executed by the one or more processors, cause the one or more processors to perform operations comprising:
receiving sensor data from the sensor array;
determining, from the sensor data, a coverage of the sensor array relative to the tympanic membrane;
determining, from the sensor data, whether an occlusion area within the ear canal impacts the coverage of the sensor array;
mapping a temperature distribution of temperatures of the tympanic membrane utilizing the sensor data, wherein the mapping is performed when the coverage satisfies a coverage criterion and is based at least in part on sensor data for which the occlusion area has been excluded or compensated;
detecting surrounding temperatures of surrounding tissues in the ear canal;
generating temperature measurement differentials as between the temperatures of the tympanic membrane and the surrounding temperatures;
detecting, from the temperature distribution and the temperature measurement differentials, an abnormal heat signature caused by inflammation on the tympanic membrane;
detecting the acute otitis media based at least in part on detecting the abnormal heat signature; and
displaying, using the display unit and to the healthcare professional, the temperature distribution such that areas associated with the abnormal heat signature are visually distinguished.

2. The infrared temperature sensing device of claim 1, the operations further comprising displaying, via the display unit, a visual representation of a coverage of the sensor array within an ear canal of the patient.

3. The infrared temperature sensing device of claim 1, the operations further comprising:
receiving input data requesting activation of the sensor array; and
activating the sensor array based at least in part on the input data.

4. The infrared temperature sensing device of claim 1, wherein the sensor array is configured to operate within a predetermined temperature range.

5. The infrared temperature sensing device of claim 1, the operations further comprising detecting, from the temperature distribution, an abnormal heat signature caused by inflammation on the tympanic membrane, wherein detecting the acute otitis media is based at least in part on detecting the abnormal heat signature.

6. The infrared temperature sensing device of claim 1, the operations further comprising: detecting surrounding temperatures of surrounding tissues in an ear canal;
generating temperature measurement differentials as between the temperatures of the tympanic membrane and the surrounding temperatures; and
wherein detecting the acute otitis media is based at least in part on the temperature measurement differentials.

7. The infrared temperature sensing device of claim 1, the operations further comprising:
detecting ambient temperatures proximate to the tympanic membrane;
generating temperature measurement differentials as between the ambient temperatures and the temperatures of the tympanic membrane; and
wherein detecting the acute otitis media is based at least in part on the temperature measurement differentials.

8. A method for detecting an acute otitis media ear infection, the method comprising:
receiving, at a device operated by a healthcare professional, sensor data from a sensor array of the device, the device including a housing having a distal end configured for at least partial insertion into an ear canal of a patient;
determining, from the sensor data, a coverage of the sensor array relative to a tympanic membrane of the patient;
determining, from the sensor data, whether an occlusion area within the ear canal impacts the coverage of the sensor array;
mapping, at the device, a temperature distribution of temperatures of the tympanic membrane utilizing the sensor data, wherein the mapping is performed when the coverage satisfies a coverage criterion and is based at least in part on sensor data for which the occlusion area has been excluded or compensated;
detecting surrounding temperatures of surrounding tissues in the ear canal;
generating temperature measurement differentials as between the temperatures of the tympanic membrane and the surrounding temperatures;
detecting, from the temperature distribution and the temperature measurement differentials, an abnormal heat signature caused by inflammation on the tympanic membrane;
detecting the acute otitis media ear infection based at least in part on detecting the abnormal heat signature; and
displaying, using a display unit of the device and to the healthcare professional, the temperature distribution such that areas associated with the abnormal heat signature are visually distinguished.

9. The method of claim 8, further comprising detecting infrared radiation corresponding to elevated temperatures indicative of infection in an ear canal, wherein detecting the acute otitis media ear infection is based at least in part on detecting the infrared radiation corresponding to the elevated temperatures.

10. The method of claim 8, further comprising tuning the sensor array to detect infrared radiation emitted from an inflamed tympanic membrane compared to tissues surrounding an ear canal.

11. The method of claim 8, further comprising:
storing trend data indicating heat patterns historically caused by the acute otitis media ear infection; and
utilizing the trend data along with the sensor data to detect the acute otitis media ear infection.

12. The method of claim 8, further comprising:
storing specific infrared signatures associated with infections in an ear canal; and
utilizing the specific infrared signatures along with the sensor data to detect the acute otitis media ear infection.

13. The method of claim 8, further comprising:
identifying a heat variation indicative of the acute otitis media ear infection;
identifying an infrared radiation variation indicative of the acute otitis media ear infection;
detecting, from the sensor data, the heat variation and the infrared radiation variation; and
wherein detecting the acute otitis media ear infection is based at least in part on detecting the heat variation and the infrared radiation variation.

14. The method of claim 8, further comprising calibrating the sensor array to generate calibrated temperature measurements, and detecting the acute otitis media ear infection by identifying, from the calibrated temperature measurements, a predefined increase in temperature in the ear canal.

15. The method of claim 8, further comprising sending notification data to another device operated by the healthcare professional, the notification data alerting the healthcare professional that the acute otitis media ear infection has been detected by the device.

16. A device of a healthcare professional, the device comprising:
one or more processors;
a sensor array configured to measure tympanic temperatures across multiple points on a tympanic membrane of a patient;
a display unit;
a housing configured to be held by the healthcare professional and to contain the one or more processors and the sensor array, the housing having a distal end configured for at least partial insertion into an ear canal of the patient; and
non-transitory computer-readable media, housed in the housing. storing instructions that, when executed by the one or more processors, cause the one or more processors to perform operations comprising:
receiving sensor data from the sensor array;
determining, from the sensor data, a coverage of the sensor array relative to the tympanic membrane;
determining, from the sensor data, whether an occlusion area within the ear canal impacts the coverage of the sensor array;
mapping a temperature distribution of temperatures of the tympanic membrane utilizing the sensor data, wherein the mapping is performed when the coverage satisfies a coverage criterion and is based at least in part on sensor data for which the occlusion area has been excluded or compensated; and
detecting surrounding temperatures of surrounding tissues in the ear canal;
generating temperature measurement differentials as between the temperatures of the tympanic membrane and the surrounding temperatures;
detecting, from the temperature distribution and the temperature measurement differentials, an abnormal heat signature caused by inflammation on the tympanic membrane; and
detecting acute otitis media based at least in part on detecting the abnormal heat signature.

17. The device of claim 16, the operations further comprising displaying, using the display unit and to the healthcare professional, the temperature distribution such that areas of abnormal heat indicative of ear infections are highlighted.

18. The device of claim 16, the operations further comprising:
determining, from first sensor data associated with a first ear of the patient, a first coverage of the sensor array relative to a first tympanic membrane and whether a first occlusion area impacts the first coverage;
determining, from second sensor data associated with a second ear of the patient, a second coverage of the sensor array relative to a second tympanic membrane and whether a second occlusion area impacts the second coverage;
excluding or compensating sensor data associated with the first occlusion area, the second occlusion area, or coverage that fails to satisfy a coverage criterion;
associating a first portion of the excluded or compensated first sensor data with a first temperature reading from a first ear of the patient;
associating a second portion of the excluded or compensated second sensor data with a second temperature reading from a second ear of the patient;
comparing the first temperature reading and the second temperature reading to detect a thermal asymmetry satisfying a threshold difference criterion; and
wherein detecting the acute otitis media is based at least in part on detecting the thermal asymmetry satisfying the threshold difference criterion after excluding or compensating the sensor data.

19. The device of claim 16, the operations further comprising:
storing trend data associated with ear infections of patients of various age ranges;
receiving input data indicating an age of the patient;
determining a subset of the trend data to utilize based at least in part on the age of the patient; and
wherein detecting the acute otitis media is based at least in part on the subset of the trend data.

20. The device of claim 16, the operations further comprising displaying, via the display unit, a color-coded alert system indicating presence or absence of the acute otitis media.
